(19) United States
(12) Patent Application Publication
(10) Pub. No.: US 2022/0203029 A1
(43) Pub. Date: Jun. 30, 2022

(54) SYSTEM AND METHOD FOR ARTIFICIAL PANCREAS WITH MULTI-STAGE MODEL PREDICTIVE CONTROL

(71) Applicant: UNIVERSITY OF VIRGINIA PATENT FOUNDATION, Charlottesville, VA (US)

(72) Inventors: Marc D. Breton, Charlottesville, VA (US); Jose Garcia-Tirado, Charlottesville, VA (US); Patricio Colmegna, Charlottesville, VA (US); John Corbett, Charlottesville, VA (US)

(21) Appl. No.: 17/611,323

(22) PCT Filed: May 14, 2020
(86) PCT No.: PCT/US2020/032855

§ 371 Date: Nov. 15, 2021

Related U.S. Application Data
(60) Provisional application No. 62/847,714, filed on May 14, 2019.
(60) Provisional application No. 62/873,066, filed on Jul. 11, 2019.
(60) Provisional application No. 62/884,479, filed on Aug. 8, 2019.

(51) Int. Cl. A61M 5/172; G16H 50/20; A61B 5/00; A61B 5/145; G16H 20/17

(52) U.S. Cl. CPC A61M 5/1723; G16H 50/20; A61B 5/7275; A61M 2230/201; A61B 5/14532; G16H 20/17; A61M 2205/702; A61B 5/4836

(57) ABSTRACT

Provided are a system and method for an artificial pancreas having multi-stage model predictive control to minimize and/or prevent occurrence of hypoglycemia associated with Type 1 diabetes. The control implements predictive modeling of a probability of glucose uptake associated with exercise based on at least one exercise profile for a subject with Type 1 diabetes. Based on the probability, the control implements an automatic adjustment of basal insulin infusion to counteract a risk of exercise-induced hypoglycemia in advance of the subject engaging in the exercise. The control implements adjustment of such infusion based on real-time signaling of exercise likely to induce hypoglycemia. The control implements adjustment of a meal-time bolus to account for delay in glucose uptake resulting from exercise engaged in by the subject. Consequently, the control acts to minimize and/or prevent hypoglycemia from occurring both during and immediately after exercise.

### CROSS-REFERENCE TO RELATED APPLICATIONS

This international application claims priority to and the benefit of each of U.S. Provisional Application No. 62/847,714, filed May 14, 2019; U.S. Provisional Application No. 62/873,066, filed Jul. 11, 2019; and U.S. Provisional Application No. 62/884,479 filed Aug. 8, 2019, the entire contents of each of such Applications being incorporated by reference herein.

### STATEMENT OF GOVERNMENT INTEREST

This invention was made with government support under Grant No. DK106826 awarded by The U.S. National Institutes of Health. The government has certain rights in the invention.

### FIELD OF THE DISCLOSURE

Disclosed embodiments relate to individual glucose control, and more specifically, to such control as enabled by use of an artificial pancreas (AP) aimed at minimizing and/or preventing the occurrence of hypoglycemic events during and immediately after moderate-intensity exercise.

### BACKGROUND

Type 1 diabetes mellitus (T1DM) is an autoimmune condition resulting in absolute insulin deficiency and a life-long need for exogenous insulin. Glycemic control in T1DM remains a challenge, despite the availability of modern insulin analogues, and advanced technology such as insulin pumps, continuous glucose monitoring (CGM) and artificial pancreas (AP) systems that automatically titrate insulin doses.

AP systems have become a focus of significant research and industrial development. During the past decade, studies have advanced from short-term, inpatient investigations using algorithm-driven manual control to long-term clinical trials in free-living conditions. Most AP studies show a significant reduction in glucose variability (GV), particularly overnight, and lower risk of hypoglycemia.

Yet, in spite of the consistent effort from the scientific community, meals and exercise remain the most challenging hurdles to the development of a fully automated AP enabling a reduction in instances of hypoglycemia. Physical activity is particularly challenging to account for because its effects on glucose are based on intensity, duration, and patient-specific physiology, e.g., moderate-intensity exercise is known to cause a decrease in glucose levels as opposed to high-intensity and anaerobic exercise which may cause an increase in glucose levels and hence an increased insulin requirement. Among the different types of exercise, moderate-intensity aerobic exercise poses a major challenge for glycemic control in this population as it is often associated with sharp declines in blood glucose (BG) concentration.

Current treatment guides suggest basal insulin reduction for pump users and/or carbohydrate supplementation prior to moderate exercise. A recent study showed that in order to prevent exercise related hypoglycemia, basal insulin needed to be reduced about 90-120 minutes before such exercise is begun. However, these approaches should be undertaken with caution as carbohydrate overconsumption and aggressive reduction of basal insulin levels may also lead to hyperglycemia during and after exercise.

Studies addressing different closed-loop control (CLC), i.e., AP, designs to improve glycemic control during and after exercise bouts have become increasingly prevalent. In these studies, the incorporation of additional sensors (e.g. heart rate (HR), accelerometry, etc.) for exercise detection, and the use of different control strategies have been assessed during moderate-intensity exercise (e.g., a 1-hour brisk walk, bicycling, or soccer). For example, CLC systems typically involve the pairing of a continuous glucose monitor (CGM) and a continuous subcutaneous insulin infusion (CSII) pump with dedicated software (known as a control system) embedded either in the pump, a handheld computer, or a smartphone. The controller automatically adjusts the insulin infusion rate frequently (e.g. every 5 minutes) based on past CGM values, insulin infusions, and announced meals.

Within the last few years, two hybrid closed-loop (HCL) systems have been approved in the U. S by the U.S. Food and Drug Administration (FDA), and include the Medtronic 670G, and more recently the t:slim X2 with Control-IQ. However, despite the tremendous progress of closed-loop control (CLC) systems, physical activity remains undeniably one of the major difficulties preventing a full automation in AP systems that may enable optimal BG control so as to avoid instances of hypoglycemic by particularly addressing both timing and type of physical activity such as exercise. Currently, investigational exercise-informed CLC systems rely on CGM and activity trackers to react as soon as possible to movement and/or steep BG declines but do not provide prospective actions aimed at minimizing and/or preventing instances of hypoglycemia and the need for treatment thereof which may result from engagement in activity such as moderate-intensity exercise.

In view of the above, it would be desirable to provide an AP incorporating a Multi-Stage Model Predictive Controller (MS-MPC) that addresses the minimization and/or prevention of hypoglycemia both during and immediately after an individual engages in, especially, moderate-intensity exercise.

The devices, systems, apparatuses, compositions, computer program products, non-transitory computer readable medium, models, algorithms, and methods of various embodiments disclosed herein may utilize aspects (e.g., devices, systems, apparatuses, compositions, computer program products, non-transitory computer readable medium, models, algorithms, and methods) disclosed in the following references, applications, publications and patents and which are hereby incorporated by reference herein in their entirety, and which are not admitted to be prior art with respect to embodiments herein by inclusion in this section:

A. U.S. Utility patent application Ser. No. 16/451,766, entitled “Method and System for Model-Based Tracking of Changes In Average Glycemia In Diabetes”, filed Jun. 25, 2019;

B. U.S. Utility patent application Ser. No. 14/769,638, entitled “Method and System for Model-Based Tracking of Changes In Average Glycemia In Diabetes”, filed Aug. 21, 2015; U.S. Pat. No. 10,332,615, issued Jun. 25, 2019;

C. International patent application Ser. No. PCT/US2014/017754, entitled “Method and System for Model-Based Tracking of Changes In Average Glycemia In Diabetes”, filed Feb. 21, 2014; Publication No. WO 2014/130841, Aug. 28, 2014;

D. U.S. Utility patent application Ser. No. 16/274,874, entitled “System and Method for Physical Activity Informed Drug Dosing”, filed Feb. 13, 2019;

E. U.S. Utility patent application Ser. No. 16/205,398, entitled “LQG Artificial Pancreas Control System and Related Method”, filed Nov. 30, 2018; Publication No. US-2019-0099555-A1, Apr. 4, 2019;

F. U.S. Utility patent application Serial No. 12/665,420, entitled “LQG Artificial Pancreas Control System and Related Method”, filed Dec. 18, 2009; U.S. Pat. No. 10,173,006, issued Jan. 08, 2019;

G. International patent application Ser. No. PCT/US2008/067723, entitled “LQG Artificial Pancreas Control System and Related Method”, filed Jun. 20, 2008; Publication No. WO 2008/157780, Dec. 24, 2008;

H. U.S. Utility patent application Ser. No. 16/126,879, entitled “Method, System and Computer Program Product for Evaluation of Insulin Sensitivity, Insulin/Carbohydrate Ratio, and Insulin Correction Factors in Diabetes from Self-Monitoring Data”, filed Sep. 10, 2018; Publication No. US-2019-0019571-A11, Jan. 17, 2019;

I. U.S. Utility patent application Ser. No. 12/665,149, entitled “Method, System and Computer Program Product for Evaluation of Insulin Sensitivity, Insulin/Carbohydrate Ratio, and Insulin Correction Factors in Diabetes from Self-Monitoring Data”, filed Dec. 17, 2009; Publication No. 2010/0198520, Aug. 05, 2010;

J. International Patent Application Serial No. PCT/US2008/069416, entitled “Method, System and Computer Program Product for Evaluation of Insulin Sensitivity, Insulin/Carbohydrate Ratio, and Insulin Correction Factors in Diabetes from Self-Monitoring Data”, filed July 08, 2008; Publication No. WO 2009/009528, Jan. 15, 2009;

K. U.S. Utility patent application Ser. No. 16/073,920, entitled “Method, System, and Computer Readable Medium for Virtualization of a Continuous Glucose Monitoring Trace”, filed Jul. 30, 2018; Publication No. US-2019-0043620-A1, Feb. 07, 2019;

L. International Patent Application Serial No. PCT/US2017/015616, entitled “Method, System, and Computer Readable Medium for Virtualization of a Continuous Glucose Monitoring Trace”, filed Jan. 30, 2017; Publication No. WO 2017/132663, Aug. 03, 2017;

M. U.S. Utility patent application Ser. No. 15/958,257, entitled “System, Method and Computer Readable Medium for Dynamical Tracking of the Risk for Hypoglycemia in Type 1 and Type 2 Diabetes”, filed Apr. 20, 2018; Publication No. US-2018-0366223-A, Dec. 20, 2018;

N. International Patent Application Serial No. PCT/US2016/058234, entitled “System, Method and Computer Readable Medium for Dynamical Tracking of the Risk for Hypoglycemia in Type 1 and Type 2 Diabetes”, filed Oct. 21, 2016; Publication No. WO 2017/070553, Apr. 27, 2017;

O. International Patent Application Serial No. PCT/US2018/016837, entitled “Method, System, and Computer Readable Medium for Controlling Insulin Delivery Using Retrospective Virtual Basal Rates”, filed Feb. 05, 2018; Publication No. WO 2018/144992, Aug. 09, 2018;

P. U.S. Utility patent application Ser. No. 15/866,384, entitled “Method, System and Computer Program Product for Real-Time Detection of Sensitivity Decline in Analyte Sensors”, filed Jan. 09, 2018; Publication No. US-2018-0323882-A1, Nov. 08, 2018;

Q. U.S. Utility patent application Ser. No. 14/266,612, entitled “Method, System and Computer Program Product for Real-Time Detection of Sensitivity Decline in Analyte Sensors”, filed Apr. 30, 2014; U.S. Pat. No. 9,882,660, issued Jan. 30, 2018;

R. U.S. Utility Patent Application Serial No. 13/418,305, entitled “Method, System and Computer Program Product for Real-Time Detection of Sensitivity Decline in Analyte Sensors”, filed Mar. 12, 2012; U.S. Pat. No. 8,718,958, issued May 06, 2014;

S. International Patent Application Serial No. PCT/US2007/082744, entitled “Method, System and Computer Program Product for Real-Time Detection of Sensitivity Decline in Analyte Sensors”, filed Oct. 26, 2007; Publication No. WO/2008/052199, May 02, 2008;

T. U.S. Utility patent application Ser. No. 11/925,689, entitled “Method, System and Computer Program Product for Real-Time Detection of Sensitivity Decline in Analyte Sensors”, filed Oct. 26, 2007; U.S. Pat. No. 8,135,548, issued Mar. 13, 2012;

U. U.S. Utility patent application Ser. No. 15/580,935, entitled “Insulin Monitoring and Delivery System and Method for CGM Based Fault Detection and Mitigation Via Metabolic State Tracking”, filed Dec. 08, 2017;

V. International Patent Application Serial No. PCT/US2016/036729, entitled “Insulin Monitoring and Delivery System and Method for CGM Based Fault Detection and Mitigation Via Metabolic State Tracking”, filed Jun. 09, 2016; Publication No. WO 2016/201120, Dec. 15, 2016;

W. U.S. Utility Patent application Ser. No. 15/580,915, entitled “System and Method for Tracking Changes in Average Glycemia in Diabetics”, filed Dec. 8, 2017; Publication No. US-2018-0313815-A1, Nov. 01, 2018; X. International Patent Application Serial No. PCT/US2016/036481, entitled “System and Method for Tracking Changes in Average Glycemia in Diabetics”, filed Jun. 8, 2016; Publication No. WO2016200970, Dec. 15, 2016;

Y. U.S. Utility patent application Ser. No. 15/669,111, entitled “Method, System and Computer Product for CGM-Based Prevention of Hypoglycemia Via Hypoglycemia Risk Assessment and Smooth Reduction Insulin Delivery”, filed Aug. 04, 2017; Publication No. US-2017-0337348-A1, Nov. 23, 2017;

Z. U.S. Utility patent application Ser. No. 14/015,831, entitled “CGM-Based Prevention of Hypoglycemia Via Hypoglycemia Risk Assessment and Smooth Reduction of Insulin Delivery”, filed Aug. 30, 2013; U.S. Pat. No. 9,750,438, issued Sep. 5, 2017;

AA. U.S. Utility patent application Ser. No. 13/203,469, entitled “CGM-Based Prevention of Hypoglycemia via Hypoglycemia Risk Assessment and Smooth Reduction Insulin Delivery”, filed Aug. 25, 2011; U.S. Pat. No. 8,562,587, issued Oct. 22, 2013;

BB. International Patent Application Serial No. PCT/US2010/025405, entitled “CGM-Based Prevention of Hypoglycemia Via Hypoglycemia Risk Assessment and Smooth Reduction Insulin Delivery”, filed Feb. 25, 2010; Publication No. WO 2010/099313 A1,Sep. 2, 2010;

CC. International Patent Application Serial No. PCT/US2016/050109, entitled “System, Method, and Computer Readable Medium for Dynamic Insulin Sensitivity In Diabetic Pump Users”, filed Sep. 02, 2016; Publication No. WO 2017/040927, Mar. 09, 2017;

DD. U.S. Utility patent application Ser. No. 15/255,828, entitled “System, Method, and Computer Readable Medium for Dynamic Insulin Sensitivity In Diabetic Pump Users”, filed Sep. 2, 2016; Publication No. US-2017-0056591-A1, Mar. 2, 2017;

EE. U.S. Utility patent application Ser. No. 15/252,365, entitled “Method, System and Computer Readable Medium for Predictive Hypoglycemia Detection for Mild to Moderate Exercise”, filed Aug. 31, 2016; Publication No. US-2018-0055452-A1, Mar. 01, 2018;

FF. U.S. Utility patent application Ser. No. 14/902,731, entitled “Simulation of Endogenous and Exogenous Glucose/Insulin/Glucagon Interplay In Type 1 Diabetic Patients”, filed Jan. 4, 2016; U.S. Pat. No. 10,169,544, issued Jan. 1, 2019;

GG. International Patent Application Serial No. PCT/US2014/045393, entitled “Simulation of Endogenous and Exogenous Glucose/Insulin/Glucagon Interplay In Type 1 Diabetic Patients”, filed Jul. 3, 2014; Publication No. WO2015003124, Jan. 8, 2015;

HH. U.S. Utility patent application Ser. No. 14/419,375, entitled “Computer Simulation for Testing and Monitoring of Treatment Strategies for Stress Hyperglycemia”, filed Feb. 3, 2015; Publication No. 2015-0193589, Jul. 09, 2015;

II. International Patent Application Serial No. PCT/US2013/053664, entitled “Computer Simulation for Testing and Monitoring of Treatment Strategies for Stress Hyperglycemia”, filed Aug. 05, 2013; Publication No. WO 2014/022864, Feb. 06, 2014;

JJ. U.S. Utility Patent Application Serial No. 14/128,922, entitled “Unified Platform For Monitoring and Control of Blood Glucose Levels in Diabetic Patients”, filed Dec. 23, 2013; Publication No. 2015/0018633, Jan. 15, 2015;

KK. International Patent Application Serial No. PCT/U2012/043910, entitled “Unified Platform For Monitoring and Control of Blood Glucose Levels in Diabetic Patients”, filed June. 23, 2012; Publication No. WO 2012/178134, Dec. 27, 2012;

LL. U.S. Utility patent application Ser. No. 14/128,811, entitled

“Methods and Apparatus for Modular Power Management and Protection of Critical Services in Ambulatory Medical Devices”, filed Dec. 23, 2013; U.S. Pat. No. 9,430,022, issued Aug. 30, 2016;

MM. International Patent Application Serial No. PCT/US2012/043883, entitled “Methods and Apparatus for Modular Power Management and Protection of Critical Services in Ambulatory Medical Devices”, filed Jun. 22, 2012; Publication No. WO 2012/178113, Dec. 27, 2012;

NN. U.S. Utility patent application Ser. No. 13/637,359, entitled “Method, System, and Computer Program Product for Improving the Accuracy of Glucose Sensors Using Insulin Delivery Observation In Diabetes”, filed Sep. 25, 2012; U.S. Pat. No. 9,398,869, issued Jul. 26, 2016;

OO. International Patent Application Serial No. PCT/US2011/029793, entitled “Method, System, and Computer Program Product for Improving the Accuracy of Glucose Sensors Using Insulin Delivery Observation In Diabetes”, filed Mar. 24, 2011; Publication No. WO 2011/119832, Sep. 29, 2011;

PP. U.S. Utility patent application Ser. No. 13/634,040, entitled “Method and System for the Safety, Analysis, and Supervision of Insulin Pump Action and Other Modes of Insulin Delivery in Diabetes”, filed Sep. 11, 2012; Publication No. 2013/0116649, May 09, 2013;

QQ. International Patent Application Serial No. PCT/US2011/028163, entitled “Method and System for the Safety, Analysis, and Supervision of Insulin Pump Action and Other Modes of Insulin Delivery in Diabetes”, filed Mar. 11, 2011; Publication No. WO 2011/112974, Sep. 15, 2011; RR. U.S. Utility patent application Ser. No. 13/394,091, entitled

“Tracking the Probability for Imminent Hypoglycemia in Diabetes from Self-Monitoring Blood Glucose (SMBG) Data”, filed Mar. 2, 2012; Publication No. 2012/0191361, Jul. 26, 2012;

SS. International Patent Application Serial No. PCT/US2010/047711, entitled “Tracking the Probability for Imminent Hypoglycemia in Diabetes from Self-Monitoring Blood Glucose (SMBG) Data”, filed Sep. 2, 2010; Publication No. WO 2011/028925, Mar. 10, 2011;

TT. U.S. Utility patent application Ser. No. 13/322,943, entitled “System Coordinator and Modular Architecture for Open-Loop and Closed-Loop Control of Diabetes”, filed Nov. 29, 2011; Publication No. 2012/0078067, Mar. 29, 2012;

UU. International Patent Application Serial No. PCT/US2010/036629, entitled “System Coordinator and Modular Architecture for Open-Loop and Closed-Loop Control of Diabetes”, filed May 28, 2010; Publication No. WO 2010/138848, Dec. 2, 2010;

VV. U.S. Utility patent application Ser. No. 13/131,467, entitled “Method, System, ad Computer Program Product for Tracking of Blood Glucose Variability in Diabetes”, filed May 26, 2011; U.S. Pat. No. 9,317,657, issued Apr. 19, 2016;

WW. International Patent Application Serial No. PCT/US2009/065725, entitled “Method, System, and Computer Program Product for Tracking of Blood Glucose Variability in Diabetes”, filed Nov. 24, 2009; Publication No. WO 2010/062898, Jun. 3, 2010;

XX. U.S. Utility patent application Ser. No. 12/674,348, entitled “Method, Computer Program Product and System for Individual Assessment of Alcohol Sensitivity”, filed Feb. 19, 2010; Publication No. 2011/0264374, Oct. 27, 2011;

YY. International Patent Application Serial No. PCT/US2008/073738, entitled “Method, Computer Program Product and System for Individual Assessment of Alcohol Sensitivity”, filed Aug. 20, 2008; Publication No. WO 2009/026381, Feb. 26, 2009;

ZZ. U.S. Utility patent application Ser. No. 12/664,444, entitled “Method, System and Computer Simulation Environment for Testing of Monitoring and Control Strategies in Diabetes”, filed Dec. 14, 2009; Publication No. 2010/0179768, Jul. 15, 2010;

AAA. International Patent Application Serial No. PCT/US2008/067725, entitled “Method, System and Computer Simulation Environment for Testing of Monitoring and Control Strategies in Diabetes”, filed Jun. 20, 2008; Publication No. WO 2008/157781, Dec. 24, 2008;

BBB. U.S. Utility patent application Ser. No. 12/516,044, entitled “Method, System, and Computer Program Product for the Detection of Physical Activity by Changes in Heart Rate, Assessment of Fast Changing Metabolic States, and Applications of Closed and Open Control Loop in Diabetes”, filed May 22, 2009; U.S. Pat. No. 8,585,593, issued Nov. 19, 2013; and

CCC. International Patent Application Serial No. PCT/US2007/085588, entitled “Method, System, and Computer Program Product for the Detection of Physical Activity by Changes in Heart Rate, Assessment of Fast Changing Metabolic States, and Applications of Closed and Open Control Loop in Diabetes”, filed Nov. 27, 2007; Publication No. WO2008/067284, Jun. 05, 2008.

Herein, applicable abbreviations include the following: (T1D) Type 1 Diabetes, (CGM) Continuous Glucose Monitoring, (FDA) U.S. Food and Drug Administration, (UVA) University of Virginia, (PADOVA) University of Padova, (SOGMM) Subcutaneous Oral Glucose Minimal Model, (AP) Artificial Pancreas, (MS-MPC) Multi-stage Model Predictive Control, (rMPC) Regular Model Predictive Control, (RMSE) Root Mean Square Error, Linear Time Invariant (LTI), (MDI) Multiple Daily Injections, (GV) Glucose Variability, (CLC) Closed-Loop Control, (EGP) Endogenous Glucose Production, (JOB) Insulin On Board, and Unified Safety System (USS).

### SUMMARY

It is to be understood that both the following summary and the detailed description are exemplary and explanatory and are intended to provide further explanation of the present embodiments as claimed. Neither the summary nor the description that follows is intended to define or limit the scope of the present embodiments to the particular features mentioned in the summary or in the description. Rather, the scope of the present embodiments is defined by the appended claims.

In this regard, embodiments herein provide a MS-MPC enabled to minimize and/or prevent instances of hypoglycemia. To do so, the MS-MPC considers and incorporates each of (i) at least one exercise profile including one or more individual-specific exercise behavior patterns, (ii) anticipatory and reactive modes of operation that compensate for expected and ongoing exercise, and (iii) an exercise-aware premeal bolus responsive to the aforementioned exercise.

An embodiment may include an artificial pancreas control system for regulating insulin infusion to a subject having Type 1 diabetes to minimize and/or prevent an occurrence of hypoglycemia in response to the subject engaging in exercise, in which the system may include a prediction module configured to generate a prediction of glucose uptake for the subject, and an insulin infusion control module configured to automatically generate a rate of basal insulin infusion, based on the prediction comprising a predetermined probability of exercise being engaged in by the subject, and to cause delivery of insulin to the subject according to the generated rate to maintain a glucose level thereof within an optimal range.

Each of the prediction module and the insulin infusion module may be included in at least one controller configured to communicate with a glucose monitoring device configured to transmit glucose levels of the subject and with an insulin delivery device configured to deliver insulin to the subject according to the generated rate.

The optimal range may be between about 70 mg/dl and about 180 mg/dl.

The prediction may be based on the Subcutaneous Oral Glucose Minimal Model.

The prediction module may include at least one exercise profile for the subject that defines an exercise pattern.

The probability of engagement in exercise by the subject may be determined as being positive according to a predetermined level of glucose uptake of the subject being determined as corresponding to the at least one exercise profile.

The at least one controller may be configured to cause delivery of insulin to the subject according to the generated rate in advance of the subject engaging in the exercise pattern of the at least one exercise profile.

The insulin infusion control module may be further configured to calculate an insulin bolus according to an amount of insulin uptake resulting from exercise by the subject according to the at least one exercise profile.

The insulin infusion control module is further configured to adjust the generated rate in response to receipt of a meal announcement.

The controller may be further configured to receive real-time signaling of the engagement in exercise by the subject, and to adjust the delivery of basal insulin according to a determined glucose level received by the controller from the glucose monitoring device at the time of the signaling.

The insulin infusion control module may be further configured to calculate an insulin bolus according to an amount of insulin uptake resulting from the subject engaging in the exercise corresponding to the real-time signaling.

An embodiment may include a processor-implemented method for regulating insulin infusion to a subject having Type 1 diabetes and equipped with an insulin delivery device to minimize and/or prevent an occurrence of hypoglycemia in response to the subject engaging in exercise, in which the method includes generating a dynamic model to predict glucose uptake for the subject, the model including at least one exercise profile for the subject that defines an exercise pattern therefor, assigning a predetermined level of glucose uptake to the at least one exercise profile, interpreting the dynamic model to determine whether the dynamic model includes a probability of the subject engaging in exercise according to the at least one exercise profile, determining a glucose level of the subject based on readings generated by a glucose monitoring device in communication with the subject, and if the probability is positive, automatically adjusting a basal insulin infusion rate, via the insulin delivery device, to be within an optimal range.

In the method, the glucose monitoring device may be a continuous glucose monitoring device.

In the method, the optimal range may be between about 70 mg/dl and about 180 mg/dl.

In the method, the adjusting may satisfy a cost function that weights a spread between amounts of two consecutive basal insulin injections.

In the method, the adjusting may satisfy a cost function that weights a spread between a current glucose value and a future glucose value corresponding to the predetermined level of glucose uptake.

In the method, the cost function may apply a penalty for a glucose value corresponding to hypoglycemia.

In the method, the dynamic model may be generated using a Kalman filter methodology.

In the method, the processor may be programmable to communicate with the insulin delivery device in a closed-loop or an open-loop.

The method may further include adjusting the basal insulin infusion rate in response to the processor receiving a meal announcement.

The method may further include calculating an insulin bolus according to an amount of insulin uptake resulting from the engagement in exercise by the subject.

In the method, the processor may be further configured to receive real-time signaling of the engagement in exercise by the subject, and to adjust the delivery of basal insulin according to a determined glucose level received by the processor from the glucose monitoring device at the time of the signaling.

In the method, a plurality of processors may automatically adjust the basal insulin infusion rate, via the insulin delivery device, to be within the optimal range.

An embodiment may include a non-transitory computer readable medium having stored thereon computer readable instructions to perform the aforementioned method as described above.

In certain embodiments, the disclosed embodiments may include one or more of the features described herein.

### BRIEF DESCRIPTION OF THE DRAWINGS

The accompanying drawings, which are incorporated herein and form a part of the specification, illustrate exemplary embodiments and, together with the description, further serve to enable a person skilled in the pertinent art to make and use these embodiments and others that will be apparent to those skilled in the art. Embodiments herein will be more particularly described in conjunction with the following drawings wherein:

FIG. 1 illustrates, for an individual in silico subject, exemplary results of BG profile generated using the UVA/Padova simulator compared to such BG profile as indicated by a Subcutaneous Oral Glucose Minimal Model (SOGMM), and wherein insulin boluses and basal pattern are shown;

FIG. 2 illustrates a mean glucose infusion rate (G̅I̅R̅), for all in silico subjects, of the MS-MPC when compared with an instance in which the SOGMM incorporates a UVA/Padova provided exercise bout for each of such subjects, together with an associated impulse response when such exercise bout is introduced;

FIG. 3 illustrates a timeline of an in silico protocol to be implemented according to the MS-MPC;

FIG. 4 illustrates clustering of glucose uptake signals over 30 days of exercise by an in silico subject;

FIG. 5 illustrates a comparison of operation among the MS-MPC and the rMPC, relative to an individual in silico subject;

FIG. 6 illustrates a comparison of operation among the MS-MPC and the rMPC, relative to a grouping of in silico subjects;

FIG. 7 illustrates a high level block diagram of the MS-MPC environment according to embodiments herein;

FIG. 8A illustrates an exemplary computing device which may implement the

MS-MPC;

FIG. 8B illustrates a network system which may implement and/or be used in the implementation of the MS-MPC;

FIG. 9 illustrates a block diagram which may implement and/or be used in the implementation of the MS-MPC in association with a connection to the Internet;

FIG. 10 illustrates a system which may implement and/or be used in the implementation of the MS-MPC in accordance with one or more of a clinical setting and a connection to the Internet; and

FIG. 11 illustrates an exemplary architecture embodying the MS-MPC.

### DETAILED DESCRIPTION

The present disclosure will now be described in terms of various exemplary embodiments. This specification discloses one or more embodiments that incorporate features of the present embodiments. The embodiment(s) described, and references in the specification to “one embodiment”, “an embodiment”, “an example embodiment”, etc., indicate that the embodiment(s) described may include a particular feature, structure, or characteristic. Such phrases are not necessarily referring to the same embodiment. The skilled artisan will appreciate that a particular feature, structure, or characteristic described in connection with one embodiment is not necessarily limited to that embodiment but typically has relevance and applicability to one or more other embodiments.

In the several figures, like reference numerals may be used for like elements having like functions even in different drawings. The embodiments described, and their detailed construction and elements, are merely provided to assist in a comprehensive understanding of the present embodiments. Thus, it is apparent that the present embodiments can be carried out in a variety of ways, and does not require any of the specific features described herein. Also, well-known functions or constructions are not described in detail since they would obscure the present embodiments with unnecessary detail.

The description is not to be taken in a limiting sense, but is made merely for the purpose of illustrating the general principles of the present embodiments, since the scope of the present embodiments are best defined by the appended claims.

It should also be noted that in some alternative implementations, the blocks in a flowchart, the communications in a sequence-diagram, the states in a state-diagram, etc., may occur out of the orders illustrated in the figures. That is, the illustrated orders of the blocks/communications/states are not intended to be limiting. Rather, the illustrated blocks/communications/states may be reordered into any suitable order, and some of the blocks/communications/states could occur simultaneously.

All definitions, as defined and used herein, should be understood to control over dictionary definitions, definitions in documents incorporated by reference, and/or ordinary meanings of the defined terms. The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of

“Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of example embodiments. The word “exemplary” is used herein to mean “serving as an example, instance, or illustration.” Any embodiment described herein as “exemplary” is not necessarily to be construed as preferred or advantageous over other embodiments. Additionally, all embodiments described herein should be considered exemplary unless otherwise stated.

It should be appreciated that any of the components or modules referred to with regards to any of the embodiments discussed herein, may be integrally or separately formed with one another. Further, redundant functions or structures of the components or modules may be implemented. Moreover, the various components may be communicated locally and/or remotely with any user/clinician/patient or machine/system/computer/processor. Moreover, the various components may be in communication via wireless and/or hardwire or other desirable and available communication means, systems and hardware. Moreover, various components and modules may be substituted with other modules or components that provide similar functions.

It should be appreciated that the device and related components discussed herein may take on all shapes along the entire continual geometric spectrum of manipulation of x, y and z planes to provide and meet the anatomical, environmental, and structural demands and operational requirements. Moreover, locations and alignments of the various components may vary as desired or required.

It should be appreciated that various sizes, dimensions, contours, rigidity, shapes, flexibility and materials of any of the components or portions of components in the various embodiments discussed throughout may be varied and utilized as desired or required.

It should be appreciated that while some dimensions are provided on the aforementioned figures, the device may constitute various sizes, dimensions, contours, rigidity, shapes, flexibility and materials as it pertains to the components or portions of components of the device, and therefore may be varied and utilized as desired or required.

Although example embodiments of the present disclosure are explained in some instances in detail herein, it is to be understood that other embodiments are contemplated. Accordingly, it is not intended that the present disclosure be limited in its scope to the details of construction and arrangement of components set forth in the following description or illustrated in the drawings. The present disclosure is capable of other embodiments and of being practiced or carried out in various ways.

Ranges may be expressed herein as from “about” or “approximately” one particular value and/or to “about” or “approximately” another particular value. When such a range is expressed, other exemplary embodiments include from the one particular value and/or to the other particular value.

In describing example embodiments, terminology will be resorted to for the sake of clarity. It is intended that each term contemplates its broadest meaning as understood by those skilled in the art and includes all technical equivalents that operate in a similar manner to accomplish a similar purpose. It is also to be understood that the mention of one or more steps of a method does not preclude the presence of additional method steps or intervening method steps between those steps expressly identified. Steps of a method may be performed in a different order than those described herein without departing from the scope of the present disclosure. Similarly, it is also to be understood that the mention of one or more components in a device or system does not preclude the presence of additional components or intervening components between those components expressly identified.

It should be appreciated that as discussed herein, a subject may be a human or any animal. It should be appreciated that an animal may be a variety of any applicable type, including, but not limited thereto, mammal, veterinarian animal, livestock animal or pet type animal, etc. As an example, the animal may be a laboratory animal specifically selected to have certain characteristics similar to human (e.g., a rat, dog, pig, or monkey), etc. It should be appreciated that the subject may be any applicable human patient, for example.

Some references, which may include various patents, patent applications, and publications, are cited in a reference list and discussed in the disclosure provided herein. The citation and/or discussion of such references is provided merely to clarify the description of the present disclosure and is not an admission that any such reference is “prior art” to any aspects of the present disclosure described herein. In terms of notation, “[n]” corresponds to the n^{th} reference in the list. All references cited and discussed in this specification are incorporated herein by reference in their entireties and to the same extent as if each reference was individually incorporated by reference.

The term “about,” as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. In one aspect, the term “about” means plus or minus 10% of the numerical value of the number with which it is being used. Therefore, about 50% means in the range of 45%-55%. Numerical ranges recited herein by endpoints include all numbers and fractions subsumed within that range (e.g. 1 to 5 includes 1, 1.5, 2, 2.75, 3, 3.90, 4, 4.24, and 5). Similarly, numerical ranges recited herein by endpoints include subranges subsumed within that range (e.g. 1 to 5 includes 1-1.5, 1.5-2, 2-2.75, 2.75-3, 3-3.90, 3.90-4, 4-4.24, 4.24-5, 2-5, 3-5, 1-4, and 2-4). It is also to be understood that all numbers and fractions thereof are presumed to be modified by the term “about.”

In an effort to assess operation of the MS-MPC, predictions of BG were compared using the MS-MPC and a rMPC when each was implemented on a personalized version of the SOGMM. The predictions were based on 100 in silico subjects according to an FDA approved UVA/Padova simulator, including intra- and inter-subject variations. As may be understood, the SOGMM implements the following equations, including:

$$\begin{matrix}\dot{G} ( t ) = - [ {S}_{g} + X ( t ) ] G ( t ) + {S}_{g} {G}_{b} + \frac{{k}_{abs} f}{{V}_{g} BW} {Q}_{2} ( t ) + w ( t ) & ( 1 ) \\ \dot{X} ( t ) = - {p}_{2} X ( t ) + {p}_{2} {S}_{I} [ \frac{{I}_{p} ( t )}{{V}_{i} BW} - {I}_{b} ] & ( 2 ) \\ {\dot{Q}}_{1} ( t ) = - {k}_{τ} {Q}_{1} ( t ) + m ( t ) & ( 3 ) \\ {\dot{Q}}_{2} ( t ) = - {k}_{abs} {Q}_{2} ( t ) + {k}_{τ} {Q}_{1} ( t ) & ( 4 ) \\ {\dot{I}}_{sc 1} ( t ) = - {k}_{d} {I}_{sc 1} ( t ) + u ( t ) & ( 5 ) \\ {\dot{I}}_{sc 2} ( t ) = - {k}_{d} {I}_{sc 2} ( t ) + {k}_{d} {I}_{sc 1} ( t ) & ( 6 ) \\ {\dot{I}}_{p} ( t ) = - {k}_{cl} {I}_{p} ( t ) + {k}_{d} {I}_{sc 2} ( t ) , & ( 7 )\end{matrix}$$

where G represents the plasma glucose concentration output (mg/dl), X represents the proportion of insulin in the remote compartment (1/min), Qₛₜₒ₁ and Qₛₜₒ₂ represent the glucose masses in the stomach and the gut (mg), I_{sc1} and I_{sc2} represent the amounts of non-monomeric and monomeric insulin in the subcutaneous space (mU), Iₚ represents the amount of plasma insulin (mU), w represents the effect of exercise on blood glucose levels (mg/dl/min), m represents the input rate of mixed-meal carbohydrate absorption (mg/min), and u represents the exogenous insulin input (mU/min). Parameters for equations (1)-(7) are set forth in Table 1 below.

TABLE 1
Model parameters of the SOGMM.

| Symbol | Meaning | Units |
|---|---|---|
| S_{g} | Fractional glucose effectiveness | 1/min |
| V_{g} | Distribution volume of glucose | kg/dl |
| k_{abs} | Rate constant - oral glucose consumption | 1/min |
| k_{τ} | Time constant related with oral glucose | 1/min |
| | absorption | |
| p₂ | Rate constant of the remote insulin compartment | 1/min |
| f | Fraction of intestinal absorption | — |
| V₁ | Distribution volume of insulin | 1/kg |
| k_{cl} | Rate constant of subcutaneous insulin transport | 1/min |
| k_{d} | Rate constant of subcutaneous insulin transport | 1/min |
| S₁ | Insulin sensitivity | 1/min/mU/l |
| BW | Body weight | Kg |
| G_{b} | Basal glucose concentration | mg/dl |
| I_{b} | Basal insulin concentration | mU/1 |

As may be understood, particular parameters may be fixed using a priori information, e.g., BW may be easily measured, f may be set to 0.9, and G_{b} may be estimated from the patient's most recent glycated hemoglobin, as illustrated according to equation (8) below, in which

$${G}_{b} = 28.7 \cdot HbA 1 c - 46.7 . \qquad ( 8 )$$

I_{b} may be computed from the basal infusion rate μ=μ_{b}, according to equation (9) below, in which

$${I}_{b} = \frac{{u}_{b}}{BW {k}_{cl} {V}_{i}} . \qquad ( 9 )$$

Synthetic glucose measurements for model identification were generated for each of the 100 in silico subjects, according to 10 days of data collection considering intra-patient and inter-day variability, based on 3 meals per day. It will be understood that because each in silico subject may be associated with a particular G_{b}, equation (8) was not implemented.

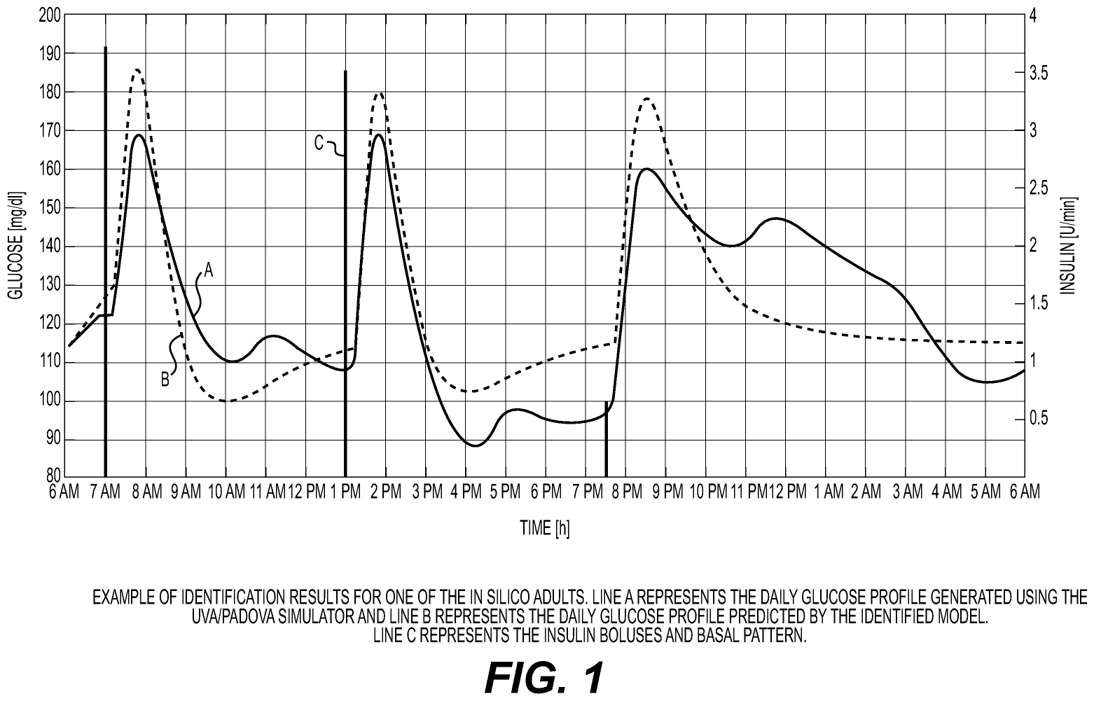

A subset of parameters was selected as θ={S_{g}, S_{I}, Vᵢ, k_{d}}. Exemplary BG for an individual subject is shown in FIG. 1, wherein line “A” indicates a daily glucose profile generated by the aforementioned simulator, and line “B” indicates the daily glucose profile as predicted by the SOGMM model. Lines “C” indicate insulin boluses and basal pattern. Performance with respect to each of the profiles was assessed by means of the root mean square error (RMSE) criterion, according to equation (10), as set forth below:

$$RMSE = \frac{‖ \hat{y} - y ‖}{\sqrt{N}} , \qquad ( 10 )$$

where indicates the 2-norm, and N, y and ŷ are the number of data points, the CGM measurements, and model output, respectively. In this regard, N was set to 288 as daily profiles, with a sampling time of 5-min. Average RMSE results considering all 1000 model identifications (10 identifications per each of the 100 virtual subjects) was determined as 14.5±6.6 mg/dl. Identified values for the population according to 0, are shown in Table 2 below.

TABLE 2
Average estimates from in silico data for
the selected parameters of the SOGMM.

| Parameter | Mean (SD) | Units |
|---|---|---|
| S_{g} | 0.0265 (0.0092) | 1/min |
| V₁ | 0.0442 (0.0250) | 1/kg |
| k_{d} | 0.1460 (0.0980) | 1/min |
| S₁ | 1.6784 × 10⁻⁴ (1.4305 × 10⁻⁴) | 1/min/mU/l |

In order to define the prediction model used by a MS-MPC controller, as well as by a rMPC controller, mean values of the 10 sets of daily parameters related to each in silico subject were implemented.

Generally, MS-MPC was introduced as a way to make the MPC strategy robust for cases where the prediction model may be uncertain, but less conservative than classic approaches. Doing so assumes a tree of semi-independent disturbance realizations which may only be related, initially, by means of a so-called non-anticipativity constraint. Such a formulation makes it possible to include further insight of what may happen in the future. As such, future control actions may be adapted according to hypothetical future realizations of the uncertainty.

With respect to the MS-MPC according to embodiments herein, the effect of a moderate-intensity exercise bout on glucose dynamics may be considered as the main source of uncertainty, i.e., a disturbance realization (Nₑₙ), in the prediction model. In particular, the disturbance realization Nₑₙ may indicate a level of glucose uptake. Since the user is not expected to exercise at the exact same time and for the same duration, different exercise realizations may arise. Instead of optimizing insulin infusion for a given exercise condition, a specific number of Nₑₙ may be considered. Although a higher Nₑₙ may lead to better disturbance characterization, such higher number may also pose a large computational burden. Accordingly, an optimal number of Nₑₙ may be selectively chosen according to a particular device which may be designated to implement the MS-MPC.

In an effort to assess the impact of exercise on predictions to be provided by the MS-MPC, the SOGMM was modified, via the UVA/Padova simulator, to include exercise input (w). In this regard, w included an exercise model having acknowledged exercise-related alterations in insulin-independent glucose uptake, EGP, and insulin sensitivity (Si). The model was formulated via recreating a euglycemic clamp study in the presence of a 45-minute moderate exercise bout within the simulator for the complete subject cohort, and obtaining glucose infusion rates (GIR) that closely resemble the results of a study where a similar protocol was conducted in vivo. Then, the mean GIR across all subjects (G̅I̅R̅) was computed and the following linear time-invariant (LTI) system was derived to describe its biphasic behavior, according to equation (11), in which

$$\begin{matrix}E ( s ) = {E}_{f} ( s ) + {E}_{s} ( s ) \\ = \frac{{k}_{1}}{( s + {p}_{11} ) ( s + {p}_{12} )} + \frac{{k}_{1}}{( s + {p}_{21} ) ( s + {p}_{22} ) ( s + {p}_{23} )} {e}^{- τ s} .\end{matrix} \qquad ( 11 )$$

E(s) may be defined as the combination of two transfer functions, E_{f}(s) and Eₛ(s), that describe the immediate glucose requirement associated with exercise as well as the delayed glucose uptake associated with the exercise (where τ375 min). The continuous-time model E(s) was converted to a discrete-time model E(z), considering the controller sampling time tₛ=5 min, and identified on G̅I̅R̅ (with 91.9% fitting), using the adaptive subspace Gauss-Newton search. In this way, given a d-minute exercise signal, π_{d,k} may be defined as follows:

$${π}_{d , k} = \{ \begin{matrix}1 & if {t}_{k} ∈ [ {t}_{ex} , {t}_{ex} + d ] \\ 0 & otherwise\end{matrix} ,$$

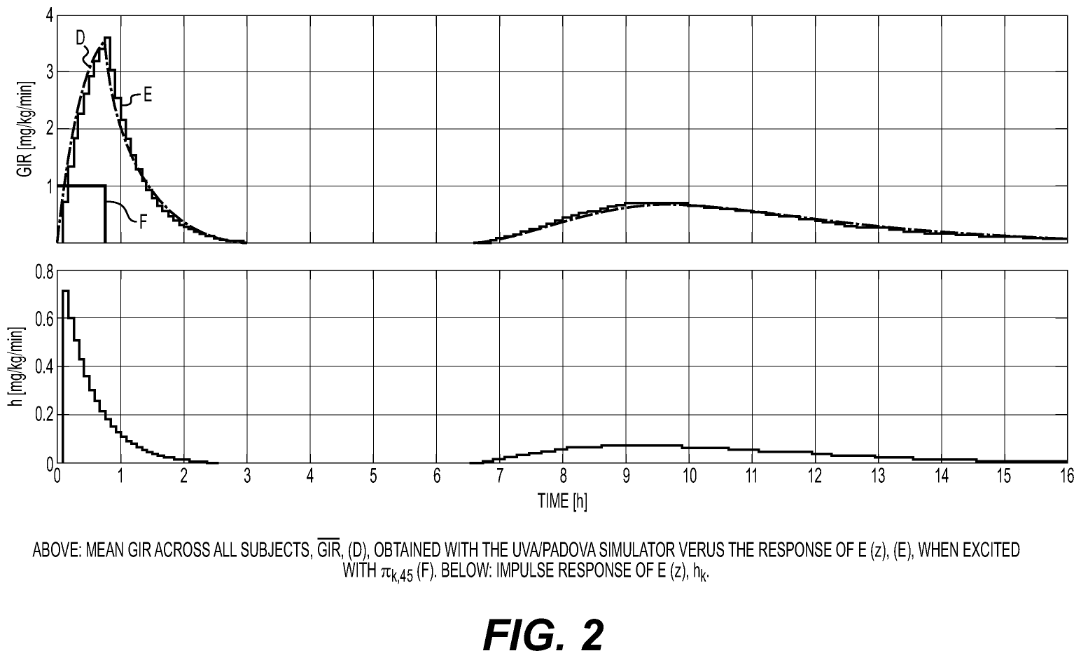

with tₑₓ defining the exercise start time. The disturbance signal w_{d,k} may be found through the discrete convolution of π_{d,k} and the impulse response, hₖ, of E(z), in terms of:

$${w}_{d , k} = - {\sum }_{n = - ∞}^{∞} {π}_{d , k} {h}_{k - n} \text{/} {V}_{g} ,$$

where V_{g} represents the distribution volume of glucose (dl/kg), and was fixed to 1.6 dl/kg. FIG. 2 shows the (G̅I̅R̅) across the cohort (at line “D”) versus the response of discrete-time model E(z) (at line “E”) when excited by a 45 minute exercise signal, π_{k,45} (as indicated by line “F.”)

Relative to the exercise input (w), signals thereof were clustered to inform the SOGMM. To do so, and simulate data leading up to a clinical admission, 30 days of simulated data for each of the in silico subjects was constructed. On one half the 30 simulated days, the subjects exercised for about 45 min in between 4-7 p.m., under moderate-intensity exercise training. The exercise bout was represented with a rectangular signal, π_{d,k}, equal to 1 during exercise and corresponding to the length of the activity. This was then convolved with the response of the previously described LTI system, hₖ, representing the dynamics of glucose uptake related to moderate-intensity exercise. Exercise disturbance signals were then calculated for each day of data collection through the aforementioned process.

24-hour exercise related disturbance signals were then clustered into 5 distinct groups using the k-medoids algorithm with a squared Euclidean distance measure. The clustered signals were then averaged across each sampling period to create a 24-hour profile trace for each grouping. The proportion of days of the month that fell into each cluster was considered as the relative probability of exercise for each subject, according to equation (12), in which

$$Pr ( i ) = \frac{{n}_{i}}{{\sum }_{j = 1}^{c} {n}_{j}} , \qquad ( 12 )$$

where Pr(i) is the probability of cluster i, nᵢ is the number of days in cluster i, and c is the number of total clusters (e.g., 5).

In this way, the MS-MPC may implement a prediction module in which a prediction of glucose uptake may be associated with at least one exercise profile of a subject. That is, the prediction which may be generated by the prediction module may include a predetermined probability of exercise being engaged in by the subject, according to the aforementioned clustering. As such, the prediction module may render a prediction of glucose uptake that may be associated with the at least one exercise profile. Likewise, the prediction of glucose uptake may be predetermined so as to correspond to the predetermined probability of exercise. It is to be noted that the at least one exercise profile may include at least one exercise pattern, and that the MS-MPC may be configured to consider multiple exercise profiles, e.g., at least five (5) thereof. The at least one exercise pattern may be derived from exercise input w that may be fed to the MS-MPC and/or otherwise derived from a historical record of the subject accumulated by, for example, an activity tracker such as a FITBIT CHARGE 2.

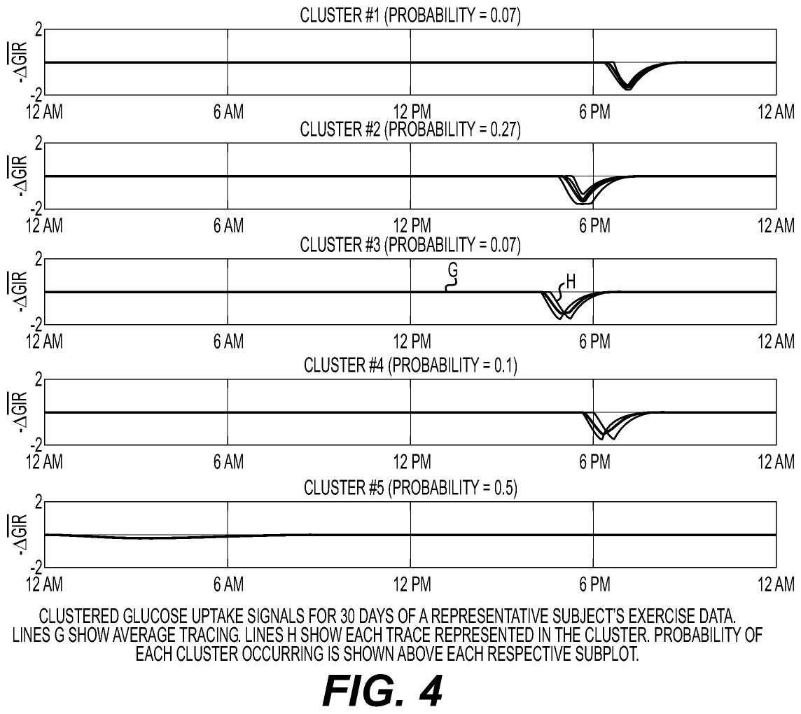

Referring to FIG. 4, there is illustrated an exemplary clustering (with an indicated probability of occurrence) for a given in silico subject, wherein an average trace is indicated by lines “G,” and each trace within a cluster is indicated by lines “H.”

In view of the above, the MS-MPC may be equipped to receive individual exercise input and extract patterning thereof so as to predict duration and frequency of such exercise. With such duration and frequency information, the MS-MPC may be further configured to act on such historical information to adjust insulin infusion in advance of when exercise will occur. Thus, if BG is predicted to deviate from the optimal range based on a probability of the at least one exercise profile occurring, the basal insulin infusion rate may be increased or decreased based on current and past CGM values, infusion trends and JOB. In an embodiment, the advance period before exercise will occur may be at least two (2) hours, and may be (i) set manually on the MS-MPC, or (ii) set within the MS-MPC as the start time for the beginning of insulin adjustment in response to the MS-MPC's prediction of a predetermined probability of the subject engaging in the at least one exercise profile. In this way, the MS-MPC replaces any reliance on preventative carbohydrate consumption and glucagon injection, which would otherwise be necessary to avoid occurrences of hypoglycemia during and immediately after moderate-intensity exercise.

More specifically, the MS-MPC may be configured to leverage the Unified Safety System (USS Virginia), a safety supervision module to limit basal injections based on the perceived risk for hypoglycemia, and implement an insulin infusion control module to assess the at least one exercise profile through analysis and resolution of the following equations (13)-(20), providing:

$$\begin{matrix}{min}_{{\tilde{u}}_{k}^{i} , {\tilde{v}}_{k}^{i}} {ϕ}^{ms} , & ( 13 ) \\ s . t . {x}_{k + j + 1 \vert k}^{i} = {Ax}_{k + j \vert k}^{i} + {B}_{I} {u}_{k + j \vert k}^{i} + {B}_{w} {w}_{k + j \vert k}^{i} , & ( 14 ) \\ {y}_{k}^{i} = {Cx}_{k}^{i} , & ( 15 ) \\ {u}_{min} \leq {u}_{k + j \vert k}^{i} \leq {u}_{max} , ∀ i = 1 , … , {N}_{en} , & ( 16 ) \\ Δ {u}_{min} \leq Δ {u}_{k + j \vert k}^{i} \leq Δ {u}_{max} , ∀ i = 1 , … , {N}_{en} , & ( 17 ) \\ {y}_{min} - {y}_{k + j}^{i} \leq {η}_{k + j}^{i} , & ( 18 ) \\ {η}_{k + j}^{i} \geq 0 , and & ( 19 ) \\ {u}_{k}^{i} = {u}_{k}^{l} with i \neq l , & ( 20 )\end{matrix}$$

where ũₖⁱ=uₖuₖ₊₁. . . u_{k+N-1}]ⁱ and e,otl nₖⁱ=[nₖnₖ₊₁ . . . n_{k+N-1}]ⁱ represent the control policy and the policy of slack variables related to the soft constraint (18) optimized at the i-th MPC with control and prediction horizons N_{c} and Nₚ, respectively, and i=1,2, . . . , Nₑₙ. The MS-MPC may be configured to resolve equations (13)-(20) at every sampling time, i.e., for every 5 minutes, of received historical data.

In the above formulation, (14) corresponds to the linear state-space representation of the i-th prediction model, with xₖⁱ ∈Rⁿ representing the system state, uₖⁱ ∈R^{m} representing the control policy, and wₖⁱ ∈R^{d} representing a specific realization of the effect of exercise on glucose dynamics, and wherein =7, m =1, and d=1. The quadruplet (A, B_{I}, B_{w}, C) may be determined after discretizing (tₛ=5 min) the matrices of the continuous-time linear approximation of equations (1)-(7) and be defined by:

$${A}_{c} = \frac{\partial g}{\partial x} {\vert}_{x = {x}_{ss} \\ u = {u}_{ss}} , {B}_{I , c} = \frac{\partial g}{\partial u} {\vert}_{x = {x}_{ss} \\ u = {u}_{ss}} , {B}_{w , c} = {[ 1 \; 0 ⋯ 0 ]}^{T} , {C}_{c} = [ 1 \; 0 ⋯ 0 ] ,$$

where xₛₛ denotes the steady state found by solving the equations (1)-(7), when considering x₁=yₛₚ=120 mg/dl, u=uₛ =u_{b}, and w=0, with u_{b} representing the subject-specific basal infusion. The model prediction for every scenario may be the same, except for receipt of an unexpected disturbance realization. Equation (15) may represent the output equation at the i-th scenario. Equations (16) and (17) ensure that both insulin infusion and the difference between two consecutive insulin infusions along a control horizon may be in the intervals [uₘᵢₙ,uₘₐₓ] and [Δuₘᵢₙ, Δuₘₐₓ], respectively, so as to account for a spread between amounts of the injections. Equations (18) and (19) together represent a soft constraint over the output's lower bound, and Equation (20) represents a non-anticipativity constraint that may prevent the MS-MPC from acting on hypothetical non-causal scenarios. The cost function for this optimization problem is defined as set forth in equation (21) below, in which:

$${ϕ}^{ms} = \frac{1}{2} {\sum }_{i = 1}^{{N}_{en}} Pr ( i ) \cdot [ {\sum }_{j = 0}^{{N}_{p - 1}} {‖ {y}_{k + j + 1 \vert k}^{i} - {r}_{k + j + 1 \vert k}^{i} ‖}_{Q}^{2} + {κ}_{1} {‖ {η}_{k + j + 1 \vert k}^{i} ‖}_{2}^{2} + {\sum }_{j = 0}^{{N}_{c - 1}} {λ}_{1} ‖ Δ {u}_{k + j \vert k}^{i} ‖ ] , \qquad ( 21 )$$

where Pr (i) denotes the probability of occurrence of scenario i=1, . . ., Nₑₙ, λ₁, and k₁ are scalar weights, and Q represents a matrix weighting the confidence on model predictions, e.g., on a difference in amount between two predicted, consecutive basal injections. In this way, Q may also represent a weighting of a spread between a current BG level and the aforementioned predetermined level of glucose uptake resulting from the subject engaging in exercise according to the at least one exercise profile. The term, k₁∥n_{k+j+1|k}ⁱ ∥₂², represents a cost or penalty value to prevent the controller from taking actions leading to low glucose levels. The cost function may further account for correction of BG to the optimal or target level of 120 mg/dl, so as to be within an optimal range of 70-180 mg/dl. A modified version of an asymmetric, time-varying, exponential reference signal may be implemented and represented by equation (22) below in which

$${r}_{k + j + 1 \vert k} = \{ \begin{matrix}( {y}_{k} - {y}_{sp} ) \cdot {e}^{- ( {t}_{k + j + 1} - {t}_{k} ) \text{/} ( {τ}_{r}^{+} )} , & {y}_{k} \geq {y}_{sp} \\ 0 , & {y}_{k} \leq {y}_{sp}\end{matrix} , \qquad ( 22 )$$

with j ∈[1, . . . , Nₚ],τᵣ⁺ it representing the time constant modulating the reference decay toward the set point, and tₖ representing the discrete time.

Each model prediction may use \hat{x}_{k|k}, representing the estimate of xₖ, as an initial condition computed by means of a hybrid implementation of a Kalman filter (KF).

In order to enhance a safety profile of the AP herein, the MS-MPC may implement a detuning strategy for Q. As seen in the above cost function, Q weights the difference of the model prediction with respect to the evolution of the MS-MPC's reference, i.e. the difference between glucose uptake indicating a probability of the subject engaging in exercise with respect to the evolution of current CGM measurements. The detuning strategy of Q may be implemented to avoid a possible overreaction to meal-induced glycemic excursions which may cause postprandial hypoglycemia. Such a detuning strategy depends on a IOB estimate relative to its basal value as follows:

$$Q ( IOB ) = \{ \begin{matrix}{Q}_{0} & if IOB < 0 \\ m \cdot IOB + {Q}_{0} & if IOB ∈ [ 0 , TDI \text{/} α ] \\ {Q}_{0} \text{/} β & if IOB > TDI \text{/} α\end{matrix} , \\ with m = \frac{α \cdot ( 1 - β ) \cdot {Q}_{0}}{β \cdot TDI} ,$$

and where TDI denotes the subject-specific total daily insulin requirement, Q₀ represents the default value of Q at the basal IOB, and α and β represent tuning parameters. The higher αand β, the less responsive the controller may be at mealtimes. Herein, Q₀, α and β may be set to 10, 20 and 1000, respectively.

By default, the MS-MPC operates in an anticipative mode to progressively reduce basal insulin infusion in response to the MS-MPC predicting a probability of exercise being engaged in by a subject according to a prediction of glucose uptake associated with the exercise. In other words, the MS-MPC does not begin the progressive reduction in basal insulin infusion at the outset of exercise being engaged in by a subject, but rather begins such reduction automatically according to its prediction of glucose uptake resulting from an identified, predetermined probability of exercise to be engaged in by a subject. As discussed, the predetermined probability of exercise may be calculated by the MS-MPC based on prior exercise activity of the individual that itself is based on a historical record of the subject, and whereby a predetermined level of glucose uptake may be learned from modeling associated with the exercise. Specifically, the MS-MPC may be configured to receive input of the prior exercise behavior and determine the at least one profile thereof including at least one pattern of exercise so as to predict, based on the at least one profile, an associated predetermined level of glucose uptake. The input may include a schedule including a particular day and time of a particular exercise. This way, the MS-MPC may minimize and/or prevent hypoglycemia from ever occurring since the advance reduction of insulin infusion accounts for the expenditure of glucose that will be associated with the impending exercise. Yet, if exercise is detected, MS-MPC may transition to a reactive mode. In the reactive mode, the MS-MPC may be configured to detect and receive real-time CGM disturbance signaling or other signaling indicating that exercise is being performed from, for example, an activity tracker configured to communicate with the MS-MPC. This allows the MS-MPC to adjust to a specific exercise bout and mitigates hypoglycemia in cases where exercise is not expected, i.e., is not probable. In other words, the reactive mode may be engaged either within or outside of the aforementioned two (2) hour advance period discussed above.

In an effort to further minimize and/or prevent instances of hypoglycemia from occurring immediately after exercise has occurred, the MS-MPC may be further configured to include an exercise-informed pre-meal bolus calculator. Such a calculator may consider the effect of previously undertaken exercise and any adjustment to basal infusion to compensate for, as previously discussed, w_{dk}, which represents an anticipated change in glucose uptake over time subsequent to performance of the exercise. Based on this quantity, the MS-MPC may be configured to calculate ΔGU_{DIA} representing the additional glucose uptake that may be anticipated to occur during the time that a meal bolus will be active (i.e., duration of insulin action—DIA). ΔGU_{DIA} may be calculated as the corresponding area under the ΔFIR curve and translated into grams as follows, according to equation (23) below:

$$Δ {GU}_{DIA} = - {\sum }_{k = t}^{t + DIA} \frac{{w}_{d , k} {V}_{G} BW}{1000} \qquad ( 23 )$$

Mealtime insulin may be computed based on carbohydrate intake, BG value at the time of the meal, IOB, and the ΔGU_{D1a}. The exercise informed bolus provided by the calculator may be obtained by correcting the standard bolus to account for the anticipated change in the glucose uptake resulting from the exercise performed prior to scheduled administration of the standard bolus as follows, according to equation (24):

$${EX}_{B , k} = \frac{CHO {Intake}_{k}}{CR} + \frac{{BG}_{k} - {BG}_{target}}{CF} - {IOB}_{k} - \frac{Δ {GU}_{DIA}}{CR} , \qquad ( 24 )$$

where CHO Intakeₖ represents an amount of ingested carbohydrates at time k, BG_{target}=yₛₚ, CR and CF represent an individual's current carbohydrate ratio and correction factors, respectively, BG represents a blood glucose sensor reading at the time of the meal, and IOB represents the current IOB from basal and correction insulin injections. The MS-MPC may calculate the BG correction component of the bolus by dividing ΔGU_{DIA} by CR, and subtracting that quantity from the standard bolus.

Thus, as will be understood, the MS-MPC may be configured to provide for a bolus adjustment upon receipt and interpretation of a disturbance signal indicating the engagement in exercise. In these ways, the standard bolus may be decreased as a result of the MS-MPC receiving only the aforementioned disturbance signal. In other words, since such decreased bolus is a function of only previously performed exercise, and the MS-MPC does not function to automatically account for a mealtime bolus, the mealtime bolus may be administered as usual according to CGM measurement.

When assessing the performance of the MS-MPC compared to the rMPC, which is not configured to either (1) account for receipt of individual-specific exercise behavior; (2) execute anticipatory and reactive modes of operation in response to expected and ongoing exercise; and (3) provide for the aforementioned exercise-informed pre-meal bolus calculator, reference may be had to Table 3 as set forth below and in which, in the context of an in silico study as discussed herein, tuning parameters for each of the MS-MPC and rMPC are provided.

TABLE 3
Tuning parameters for the rMPC and MS-MPC

| Parameter | rMPC | MS-MPC | Parameter | rMPC | MS-MPC |
|---|---|---|---|---|---|
| Nₑₙ | N.A. | 5 | τᵣ⁺ | 25 min | 25 min |
| Nₚ | 24 | 24 | uₘᵢₙ | −u_{b} | −u_{b} |
| N_{c} | 18 | 18 | Δuₘₐₓ | 50 | 50 |
| λ₁ | 1750/u_{b} | 1750/u_{b} | yₘᵢₙ | 70 | 70 |
| κ₁ | 100 | 100 | | | |

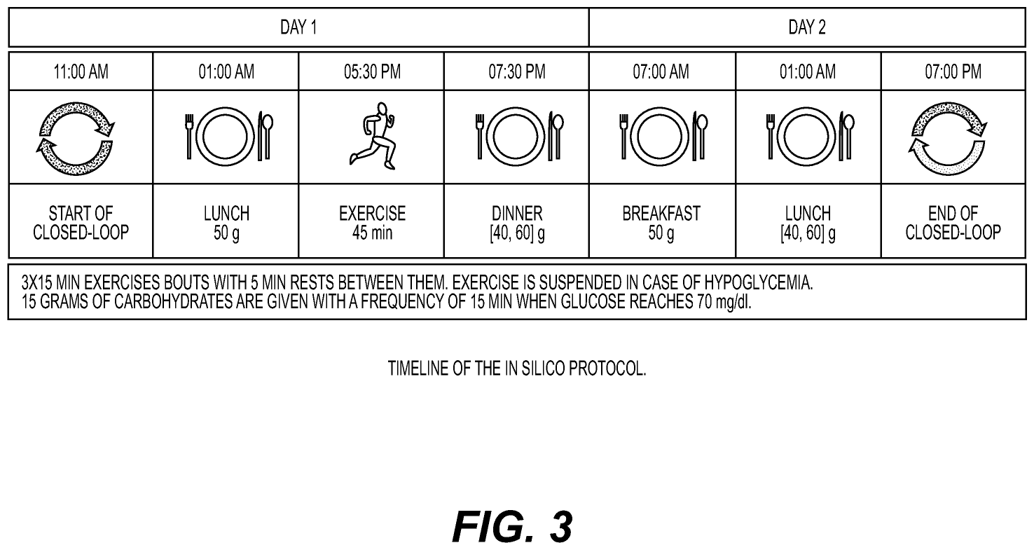

A particular regimen for the in silico comparative study may be seen with reference to FIG. 3, in which in silico participants began in a fasting state and intra- and inter-day variability in insulin sensitivity and dawn phenomenon are included. At each 5-min interval, the proposed control strategy computes a new basal insulin dose, and transmits it to an insulin pump of the in silico participant. Following the principles of hybrid closed-loop control, a manual meal bolus was administered at mealtimes. Although each in silico participant was equipped with diurnal patterns of CR and basal insulin rate, nominal basal rates were considered. Basal insulin rate that does not minimize per se glucose oscillations caused by insulin sensitivity and dawn phenomena was observed.

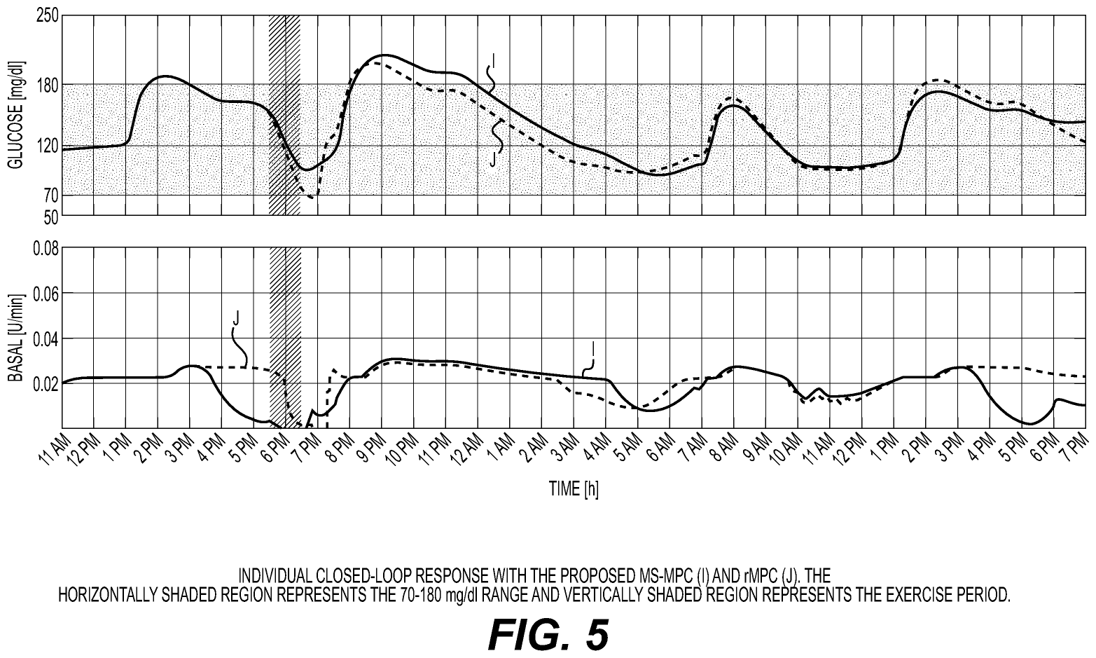

Referring to FIG. 5, there is shown an exemplary activation of the rMPC and the MS-MPC in response to the vertically shaded area representing a period of exercise. Relative to the horizontally shaded area representing a target BG range of 70-180 mg/dl, the MS-MPC performed to avoid a hypoglycemic event, as shown by line “I,” while despite essentially “turning off” the insulin pump, the rMPC could not avoid hypoglycemia from occurring, as shown by line “J.”

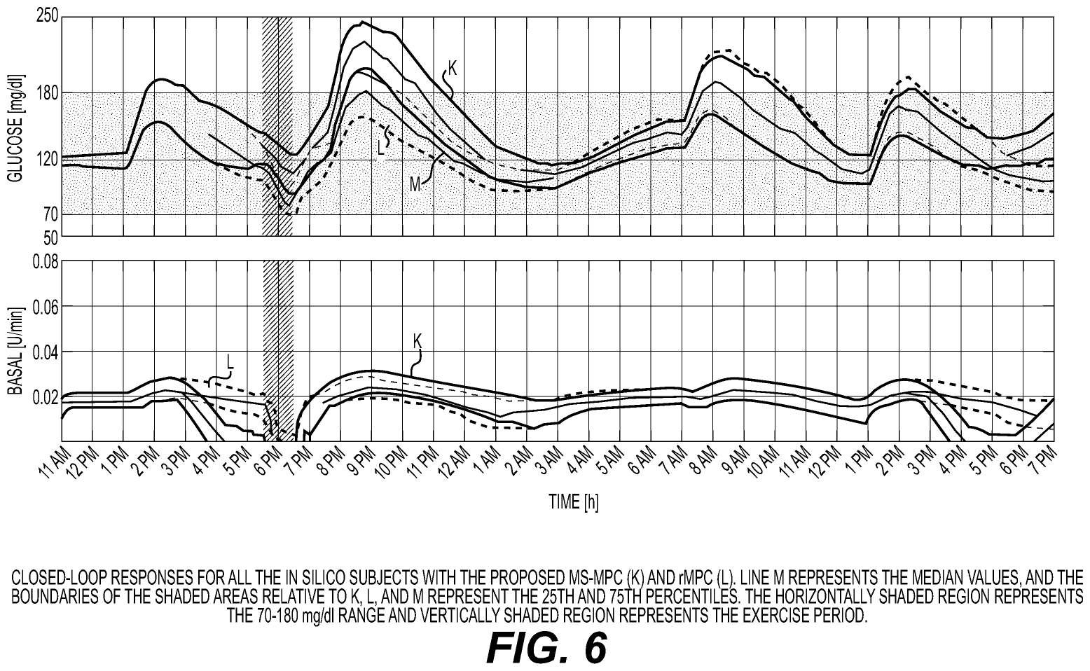

Though FIG. 5 presents results in the context of an individual in silico participant, the results as illustrated in FIG. 6 are no different with respect to the cohort of study participants.

The average closed-loop responses obtained with both the proposed MS-MPC and rMPC are compared in FIG. 6 and the average results are summarized in Table 4 below.

TABLE 4
Average closed-loop results for all the in silico
subjects with the MS-MPC and rMPC strategies.

| | MS-MPC | | | rMPC | | |
|---|---|---|---|---|---|---|
| | Mean | Median | IQR | Mean | Median | IQR |
| Average blood | 144.7 | 142.5 | 16.6 | 136.6 | 135.3 | 19.0 |
| glucose (mg/dl) | | | | | | |
| % time > 250 mg/dl | 1.66 | 0.00 | 2.34 | 0.52 | 0.00 | 0.00 |
| % time > 180 mg/dl | 18.56 | 16.10 | 15.71 | 13.66 | 11.69 | 20.26 |
| % time in | 81.16 | 83.90 | 16.49 | 85.56 | 87.92 | 20.26 |
| [70, 180] mg/dl | | | | | | |
| % time in | 54.62 | 54.55 | 21.56 | 60.38 | 58.70 | 22.99 |
| [70, 140] mg/dl | | | | | | |
| % time < 70 mg/dl | 0.28 | 0.00 | 0.52 | 0.77 | 0.78 | 1.04 |
| LBGI | 0.19 | 0.18 | 0.20 | 0.36 | 0.35 | 0.21 |
| HBGI | 3.90 | 3.44 | 2.84 | 2.94 | 2.68 | 2.63 |

| | | |
|---|---|---|
| # hypo treats during | 8 | 68 |
| exercise | | |

Safety and effectiveness endpoints based on consensus outcome metrics for glucose controllers' performances were computed for the duration of the in silico protocol. In

FIG. 6, area “K” represents performance of the MS-MPC, and area “L” represents performance of the rMPC, and wherein the vertically shaded area represents a period of exercise and the horizontally shaded area represents a target BG range of 70-180 mg/dl. With respect to the MS-MPC performance, time within the target range of 70-180 mg/dl exceeds 80%, and the primary safety parameter, the Low BG Index (LBGI), indicated minimal risk of hypoglycemia (LBGI <1.1). As expected, the MS-MPC demonstrated better performance for hypoglycemia protection during and after exercise than did the rMPC, and with less time spent in hypoglycemia. In this regard, 58 subjects received at least one hypo treatment during the exercise period and 10 subjects received 2 hypo treatments under rMPC, while only 8 received treatment when using the MS-MPC. Thus, despite occurrence of higher average glucose concentration being obtained with the MS-MPC controller, risk for hyperglycemia (HBGI<4.5) was decreased. In order to modulate the risk for hypoglycemia that may result after consumption of a meal due to delayed glucose uptake following exercise, it is contemplated that the MS-MPC may be configured to determine insulin infusion based on insulin having faster on and off pharmacodynamics.

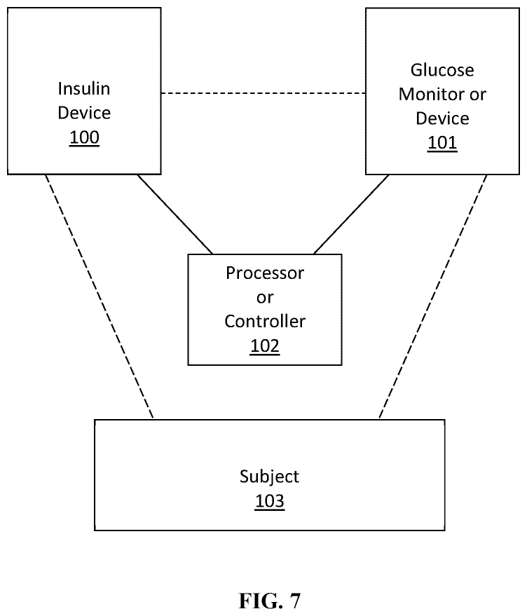

Referring to FIGS. 7-11, there are illustrated various apparatuses and associated architecture for implementing operability of the AP discussed herein and its constituent MS-MPC. In particular, and has been discussed, the MS-MPC is operable to effect a prospective manipulation of insulin infusion to decrease the incidence of exercise-induced hypoglycemia resulting from, particularly, moderate-intensity exercise. In these regards, the MS-MPC is operable to enact one or more platforms for enacting instructions to perform tasks including (i) receiving and translating updatable exercise information as a behavioral pattern to provide ongoing timely information as input to the MS-MPC, (ii) executing a probabilistic framework allowing prioritization and use of specific exercise signals based on their likelihood, and (iii) adjusting post-exercise meal boluses to account for estimated future, exercise-related glucose uptake.

Referring to FIG. 7, there is shown a high level functional block diagram of an AP according to embodiments herein.

As shown, a processor or controller 102, such as the MS-MPC herein, may be configured to implement each of the prediction module and insulin infusion control module discussed above and to communicate with a CGM 101 (such as a DEXCOM G6), and optionally with an insulin device 100 enabled to deliver insulin. The glucose monitor or device 101 may communicate with a subject 103 to monitor glucose levels thereof. The processor or controller 102 may be configured to include all necessary hardware and/or software necessary to perform the required instructions to achieve the aforementioned tasks. Optionally, the insulin device 100 may communicate with the subject 103 to deliver insulin thereto. The glucose monitor 101 and the insulin device 100 may be implemented as separate devices or as a single device in combination. The processor 102 may be implemented locally in the glucose monitor 101, the insulin device 100, or as a standalone device (or in any combination of two or more of the glucose monitor, insulin device, or a standalone device). The processor 102 or a portion of the AP may be located remotely, such that the AP may be operated as a telemedicine device.

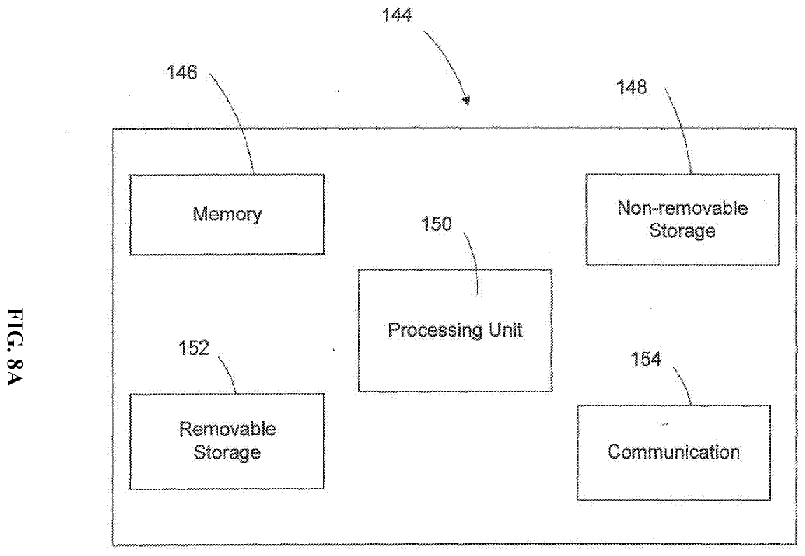

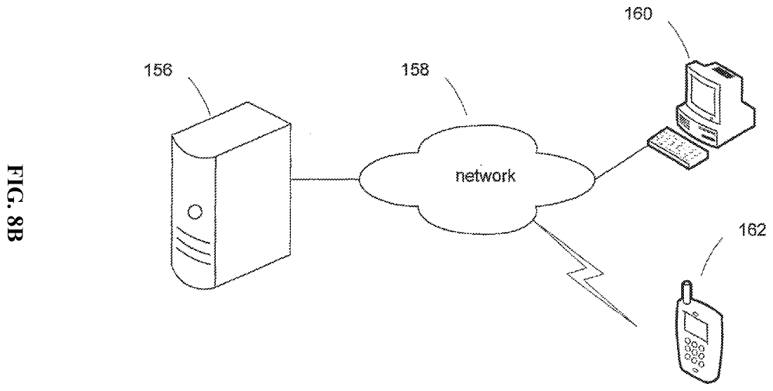

Referring to FIG. 8A, a computing device 144 may implement the MS-MPC and may typically include at least one processing unit 150 and memory 146. Depending on the exact configuration and type of computing device, memory 146 may be volatile (such as RAM), non-volatile (such as ROM, flash memory, etc.) or some combination of the two.

Additionally, computing device 144 may also have other features and/or functionality. For example, the device could also include additional removable and/or non-removable storage including, but not limited to, magnetic or optical disks or tape, as well as writable electrical storage media. Such additional storage may be represented as removable storage 152 and non-removable storage 148. Computer storage media may include volatile and nonvolatile, removable and non-removable media implemented in any method or technology for storage of information such as computer readable instructions, data structures, program modules or other data. The memory, the removable storage and the non-removable storage may comprise examples of computer storage media. Computer storage media may include, but not be limited to, RAM, ROM, EEPROM, flash memory or other memory technology CDROM, digital versatile disks (DVD) or other optical storage, magnetic cassettes, magnetic tape, magnetic disk storage or other magnetic storage devices, or any other medium which can be used to store the desired information and which can accessed by the device. Any such computer storage media may be part of, or used in conjunction with, one or more components of the AP and its MS-MPC.

The computer device 144 may also contain one or more communications connections 154 that allow the device to communicate with other devices (e.g. other computing devices). The communications connections may carry information in a communication media. Communication media may typically embody computer readable instructions, data structures, program modules or other data in a modulated data signal, such as a carrier wave or other transport mechanism and includes any information delivery media. The term “modulated data signal” may include a signal that has one or more of its characteristics set or changed in such a manner as to encode, execute, or process information in the signal. By way of example, and not limitation, communication medium may include wired media such as a wired network or direct-wired connection, and wireless media such as radio, RF, infrared and other wireless media. As discussed above, the term computer readable media as used herein may include both storage media and communication media.

In addition to a stand-alone computing machine, embodiments herein may also be implemented on a network system comprising a plurality of computing devices that may in communication via a network, such as a network with an infrastructure or an ad hoc network. The network connection may include wired connections or wireless connections. For example, FIG. 8B illustrates a network system in which embodiments herein may be implemented. In this example, the network system may comprise a computer 156 (e.g., a network server), network connection means 158 (e.g., wired and/or wireless connections), a computer terminal 160, and a PDA (e.g., a smartphone) 162 (or other handheld or portable device, such as a cell phone, laptop computer, tablet computer, GPS receiver, mp3 player, handheld video player, pocket projector, etc. or other handheld devices (or non-portable devices) with combinations of such features). In an embodiment, it should be appreciated that the module listed as 156 may implement a CGM. In an embodiment, it should be appreciated that the module listed as 156 may be a glucose monitor device, an artificial pancreas, and/or an insulin device. Any of the components shown or discussed with FIG. 8B may be multiple in number. Embodiments herein may be implemented in anyone of the aforementioned devices. For example, execution of the instructions or other desired processing may be performed on the same computing device that is anyone of 156, 160, and 162. Alternatively, an embodiment may be performed on different computing devices of the network system. For example, certain desired or required processing or execution may be performed on one of the computing devices of the network (e.g. server 156 and/or a CGM), whereas other processing and execution of the instruction can be performed at another computing device (e.g., terminal 160) of the network system, or vice versa. In fact, certain processing or execution may be performed at one computing device (e.g. server 156 and/or insulin device, artificial pancreas, or CGM); and the other processing or execution of the instructions may be performed at different computing devices that may or may not be networked. For example, such certain processing may be performed at terminal 160, while the other processing or instructions may be passed to device 162 where the instructions may be executed. This scenario may be of particular value especially when the PDA 162 device, for example, accesses the network through computer terminal 160 (or an access point in an ad hoc network). For another example, software comprising the instructions may be executed, encoded or processed according to one or more embodiments herein. The processed, encoded or executed instructions may then be distributed to customers in the form of a storage media (e.g. disk) or electronic copy.

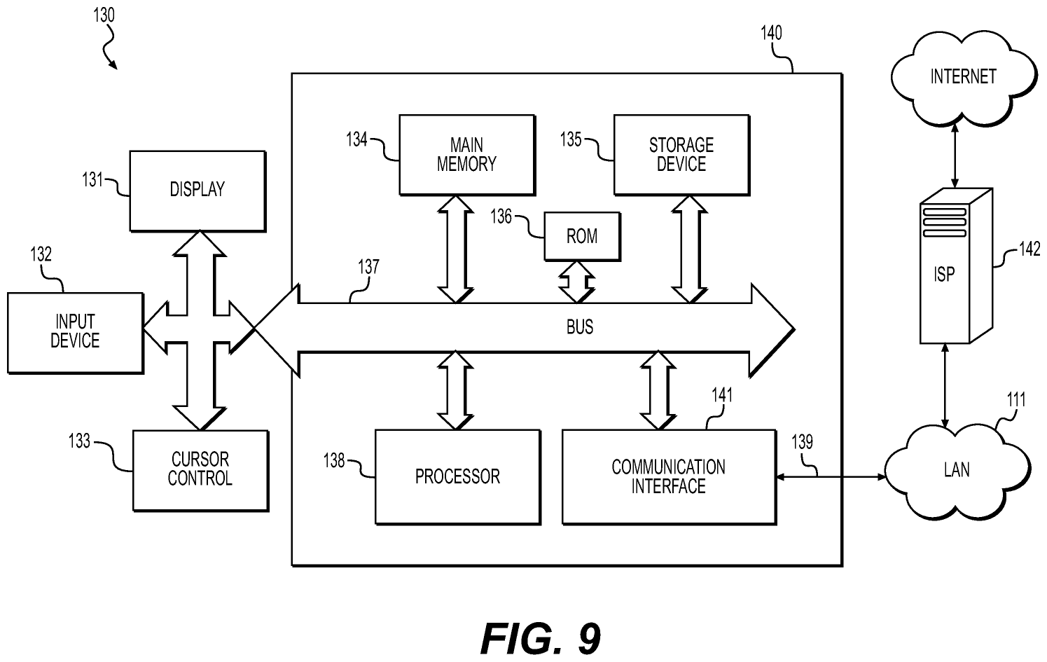

FIG. 9 illustrates a block diagram that of a system 130 including a computer system 140 and the associated Internet 11 connection upon which an embodiment may be implemented. Such configuration may typically used for computers (i.e., hosts) connected to the Internet 11 and executing software on a server or a client (or a combination thereof). A source computer such as laptop, an ultimate destination computer and relay servers, for example, as well as any computer or processor described herein, may use the computer system configuration and the Internet connection shown in FIG. 9. The system 140 may take the form of a portable electronic device such as a notebook/laptop computer, a media player (e.g., a MP3 based or video player), a cellular phone, a Personal Digital Assistant (PDA), a CGM, an AP, an insulin delivery device, an image processing device (e.g., a digital camera or video recorder), and/or any other handheld computing devices, or a combination of any of these devices. Note that while FIG. 9 illustrates various components of a computer system, it is not intended to represent any particular architecture or manner of interconnecting the components; as such, details of such interconnection are omitted. It will also be appreciated that network computers, handheld computers, cell phones and other data processing systems which have fewer components or perhaps more components may also be used. The computer system of FIG. 9 may, for example, be an Apple Macintosh computer or Power Book, or an IBM compatible PC. Computer system 140 may include a bus 137, an interconnect, or other communication mechanism for communicating information, and a processor 138, commonly in the form of an integrated circuit, coupled with bus 137 for processing information and for executing the computer executable instructions. Computer system 140 may also include a main memory 134, such as a Random Access Memory (RAM) or other dynamic storage device, coupled to bus 137 for storing information and instructions to be executed by processor 138.

Main memory 134 also may be used for storing temporary variables or other intermediate information during execution of instructions to be executed by processor 138. Computer system 140 may further include a Read Only Memory (ROM) 136 (or other non-volatile memory) or other static storage device coupled to bus 137 for storing static information and instructions for processing by processor 138. A storage device 135, such as a magnetic disk or optical disk, a hard disk drive for reading from and writing to a hard disk, a magnetic disk drive for reading from and writing to a magnetic disk, and/or an optical disk drive (such as a DVD) for reading from and writing to a removable optical disk, may be coupled to bus 137 for storing information and instructions. The hard disk drive, magnetic disk drive, and optical disk drive may be connected to the system bus by a hard disk drive interface, a magnetic disk drive interface, and an optical disk drive interface, respectively. The drives and their associated computer readable media may provide non-volatile storage of computer readable instructions, data structures, program modules and other data for the general purpose computing devices. Typically, computer system 140 may include an Operating System (OS) stored in a non-volatile storage for managing the computer resources and may provide the applications and programs with an access to the computer resources and interfaces. An operating system commonly processes system data and user input, and responds by allocating and managing tasks and internal system resources, such as controlling and allocating memory, prioritizing system requests, controlling input and output devices, facilitating networking and managing files. Non-limiting examples of OSs may include Microsoft Windows, Mac OS X, and Linux.

The term “processor” may include any integrated circuit or other electronic device (or collection of such electronic devices) capable of performing an operation on at least one instruction including, without limitation, Reduced Instruction Set Core (RISC) processors, CISC microprocessors, Microcontroller Units (MCUs), CISC-based Central Processing Units (CPUs), and Digital Signal Processors (DSPs). The hardware of such devices may be integrated onto a single substrate (e.g., a silicon “die”), or may be distributed among two or more substrates. Furthermore, various functional aspects of the processor may be implemented solely as software or firmware associated with the processor.

Computer system 140 may be coupled via bus 137 to a display 131, such as a Cathode Ray Tube (CRT), a Liquid Crystal Display (LCD), a flat screen monitor, a touch screen monitor or similar means for displaying text and graphical data to a user. The display may be connected via a video adapter for supporting the display. The display may allow a user to view, enter, and/or edit information that may be relevant to the operation of the system. An input device 132, including alphanumeric and other keys, may be coupled to bus 137 for communicating information and command selections to processor 138. Another type of user input device may include cursor control 133, such as a mouse, a trackball, or cursor direction keys for communicating direction information and command selections to processor 138, and for controlling cursor movement on display 131. Such an input device may include two degrees of freedom in two axes, a first axis (e.g., x) and a second axis (e.g., y), that may allow the device to specify positions in a plane.

The computer system 140 may be used for implementing the methods and techniques described herein. According to an embodiment, those methods and techniques may be performed by computer system 140 in response to processor 138 executing one or more sequences of one or more instructions contained in main memory 134. Such instructions may be read into main memory 134 from another computer readable medium, such as storage device 135. Execution of the sequences of instructions contained in main memory 134 may cause processor 138 to perform the process steps described herein. In alternative embodiments, hard-wired circuitry may be used in place of or in combination with software instructions to implement the arrangement. Thus, embodiments of the invention may not be limited to any specific combination of hardware circuitry and software.

The term “computer readable medium” (or “machine readable medium”) as used herein is an extensible term that refers to any medium or any memory, that participates in providing instructions to a processor, (such as processor 138), for execution, or any mechanism for storing or transmitting information in a form readable by a machine (e.g., a computer). Such a medium may store computer-executable instructions to be executed by a processing element and/or control logic, and data which may be manipulated by a processing element and/or control logic, and may take many forms, including but not limited to, non-volatile medium, volatile medium, and transmission medium. Transmission media may include coaxial cables, copper wire and fiber optics, including the wires that comprise bus 137. Transmission media may also take the form of acoustic or light waves, such as those generated during radio-wave and infrared data communications, or other form of propagated signals (e.g., carrier waves, infrared signals, digital signals, etc.). Common forms of computer readable media include, for example, a floppy disk, a flexible disk, hard disk, magnetic tape, or any other magnetic medium, a CD-ROM, any other optical medium, punch-cards, paper-tape, any other physical medium with patterns of holes, a

RAM, a PROM, and EPROM, a FLASH-EPROM, any other memory chip or cartridge, a carrier wave as described hereinafter, or any other medium from which a computer can read.

Various forms of computer readable media may be involved in carrying one or more sequences of one or more instructions to processor 138 for execution. For example, the instructions may initially be carried on a magnetic disk of a remote computer. The remote computer may load the instructions into its dynamic memory and send the instructions over a telephone line using a modem. A modem local to computer system 140 may receive the data on the telephone line and use an infra-red transmitter to convert the data to an infra-red signal. An infra-red detector may receive the data carried in the infra-red signal, and appropriate circuitry may place the data on bus 137. Bus 137 may carry the data to main memory 134, from which processor 138 may retrieve and execute the instructions. The instructions received by main memory 134 may optionally be stored on storage device 135 either before or after execution by processor 138.

Computer system 140 may also include a communication interface 141 coupled to bus 137. Communication interface 141 may provide a two-way data communication coupling to a network link 139 that may be connected to a local network 111. For example, communication interface 141 may be an Integrated Services Digital Network (ISDN) card or a modem to provide a data communication connection to a corresponding type of telephone line. As another non-limiting example, communication interface 141 may be a local area network (LAN) card to provide a data communication connection to a compatible LAN. For example, Ethernet based connection based on IEEE802.3 standard may be used such as 10/100BaseT, 1000BaseT (gigabit Ethernet), 10 gigabit Ethernet (10 GE or 10 GbE or 10 GigE per IEEE Std 802.3ae-2002 as standard), 40 Gigabit Ethernet (40 GbE), or 100 Gigabit Ethernet (100 GbE as per Ethernet standard IEEE P802.3ba), as described in Cisco Systems, Inc. Publication number 1-587005-001-3 (6/99), “Internetworking Technologies Handbook”, Chapter 7: “Ethernet Technologies”, pages 7-1 to 7-38, which is incorporated in its entirety for all purposes as if fully set forth herein. In such a case, the communication interface 141 may typically include a LAN transceiver or a modem, such as Standard Microsystems Corporation (SMSC) LAN91C111 10/100 Ethernet transceiver described in the Standard Microsystems Corporation (SMSC) data-sheet “LAN91C111 10/100 Non-PCI Ethernet Single Chip MAC+PHY” Data-Sheet, Rev. 15 (Feb. 20, 2004), which is incorporated in its entirety for all purposes as if fully set forth herein.

Wireless links may also be implemented. In any such implementation, communication interface 141 may send and receive electrical, electromagnetic or optical signals that may carry digital data streams representing various types of information. Network link 139 may typically provide data communication through one or more networks to other data devices. For example, network link 139 may provide a connection through local network 111 to a host computer or to data equipment operated by an Internet Service Provider (ISP) 142. ISP 142, in turn, may provide data communication services through the world wide packet data communication network Internet 11. Local network 111 and Internet 11 may both use electrical, electromagnetic or optical signals that carry digital data streams. The signals through the various networks and the signals on the network link 139 and through the communication interface 141, which carry the digital data to and from computer system 140, are exemplary forms of carrier waves transporting the information.

A received code may be executed by processor 138 as it is received, and/or stored in storage device 135, or other non-volatile storage for later execution. In this manner, computer system 140 may obtain application code in the form of a carrier wave.

In view of the above, minimization and/or prevention of the occurrence of hypoglycemia through use of the AP and MS-MPC discussed herein may be readily applicable into devices with (for example) limited processing power, such as glucose, insulin, and AP devices, and may be implemented and utilized with the related processors, networks, computer systems, interne, and components and functions according to the schemes disclosed herein.

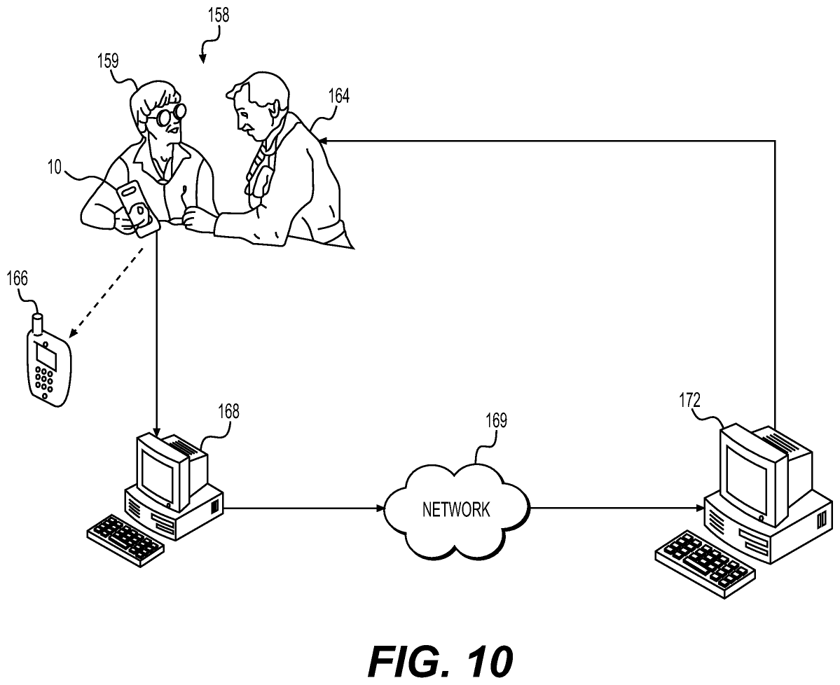

Referring to FIG. 10, there is shown an exemplary system in which examples of the invention may be implemented. In an embodiment, the CGM, the AP or the insulin device may be implemented by a subject (or patient) locally at home or at another desired location. However, in an alternative embodiment, one or more of the above may be implemented in a clinical setting. For instance, referring to FIG. 10, a clinical setup 158 may provide a place for doctors (e.g., 164) or clinician/assistant to diagnose patients (e.g., 159) with diseases related with glucose, and related diseases and conditions. A CGM 10 may be used to monitor and/or test the glucose levels of the patient—as a standalone device. It should be appreciated that while only one CGM 10 is shown in the figure, the system may include other AP components. The system or component, such as the CGM 10, may be affixed to the patient or in communication with the patient as desired or required. For example, the system or combination of components thereof—including a CGM 10 (or other related devices or systems such as a controller, and/or an AP, an insulin pump, or any other desired or required devices or components)—may be in contact, communication or affixed to the patient through tape or tubing (or other medical instruments or components) or may be in communication through wired or wireless connections. Such monitoring and/or testing may be short term (e.g., a clinical visit) or long term (e.g., a clinical stay). The CGM may output results that may be used by the doctor (, clinician or assistant) for appropriate actions, such as insulin injection or food feeding for the patient, or other appropriate actions or modeling. Alternatively, the CGM 10 may output results that may be delivered to computer terminal 168 for instant or future analyses. The delivery may be through cable or wireless or any other suitable medium. The CGM 10 output from the patient may also be delivered to a portable device, such as PDA 166. The CGM 10 output may also be delivered to a glucose monitoring center 172 for processing and/or analyzing. Such delivery can be accomplished in many ways, such as network connection 170, which may be wired or wireless.

In addition to the CGM 10 output, errors, parameters for accuracy improvements, and any accuracy related information may be delivered, such as to computer 168, and/or glucose monitoring center 172 for performing error analyses. Doing so may provide centralized monitoring of accuracy, modeling and/or accuracy enhancement for glucose centers, relative to assuring a reliable dependence upon glucose sensors.

Examples of the invention may also be implemented in a standalone computing device associated with the target glucose monitoring device. An exemplary computing device (or portions thereof) in which examples of the invention may be implemented is schematically illustrated in FIG. 8A.

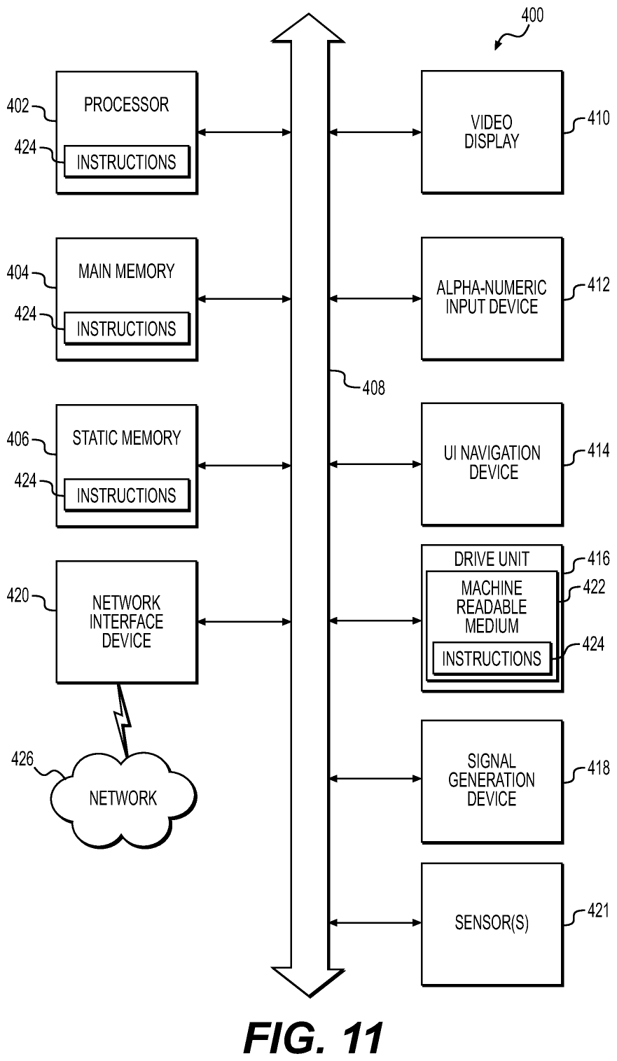

FIG. 11 provides a block diagram illustrating an exemplary machine upon which one or more aspects of embodiments, including methods thereof, herein may be implemented.

Machine 400 may include logic, one or more components, and circuits (e.g., modules). Circuits may be tangible entities configured to perform certain operations. In an example, such circuits may be arranged (e.g., internally or with respect to external entities such as other circuits) in a specified manner. In an example, one or more computer systems (e.g., a standalone, client or server computer system) or one or more hardware processors (processors) may be configured with or by software (e.g., instructions, an application portion, or an application) as a circuit that operates to perform certain operations as described herein. In an example, the software may reside (1) on a non-transitory machine readable medium or (2) in a transmission signal. In an example, the software, when executed by the underlying hardware of the circuit, may cause the circuit to perform the certain operations.

In an example, a circuit may be implemented mechanically or electronically. For example, a circuit may comprise dedicated circuitry or logic that may be specifically configured to perform one or more techniques such as are discussed above, including a special-purpose processor, a field programmable gate array

(FPGA) or an application-specific integrated circuit (ASIC). In an example, a circuit may comprise programmable logic (e.g., circuitry, as encompassed within a general-purpose processor or other programmable processor) that may be temporarily configured (e.g., by software) to perform certain operations. It will be appreciated that the decision to implement a circuit mechanically (e.g., in dedicated and permanently configured circuitry), or in temporarily configured circuitry (e.g., configured by software) may be driven by cost and time considerations.

Accordingly, the term “circuit” may be understood to encompass a tangible entity, whether physically constructed, permanently configured (e.g., hardwired), or temporarily (e.g., transitorily) configured (e.g., programmed) to operate in a specified manner or to perform specified operations. In an example, given a plurality of temporarily configured circuits, each of the circuits need not be configured or instantiated at any one instance in time. For example, where the circuits comprise a general-purpose processor configured via software, the general-purpose processor may be configured as respective different circuits at different times. Software may accordingly configure a processor, for example, to constitute a particular circuit at one instance of time and to constitute a different circuit at a different instance of time.

In an example, circuits may provide information to, and receive information from, other circuits. In this example, the circuits may be regarded as being communicatively coupled to one or more other circuits. Where multiple of such circuits exist contemporaneously, communications may be achieved through signal transmission (e.g., over appropriate circuits and buses) that connect the circuits. In embodiments in which multiple circuits are configured or instantiated at different times, communications between such circuits may be achieved, for example, through the storage and retrieval of information in memory structures to which the multiple circuits have access. For example, one circuit may perform an operation and store the output of that operation in a memory device to which it is communicatively coupled. A further circuit may then, at a later time, access the memory device to retrieve and process the stored output. In an example, circuits may be configured to initiate or receive communications with input or output devices and may operate on a collection of information.

The various operations of methods described herein may be performed, at least partially, by one or more processors that may temporarily configured (e.g., by software) or permanently configured to perform the relevant operations. Whether temporarily or permanently configured, such processors may constitute processor-implemented circuits that operate to perform one or more operations or functions. In an example, the circuits referred to herein may comprise processor-implemented circuits.

Similarly, the methods described herein may be at least partially processor-implemented. For example, at least some of the operations of a method may be performed by one or processors or processor-implemented circuits. The performance of certain of the operations may be distributed among the one or more processors, not only residing within a single machine, but deployed across a number of machines. In an example, the processor or processors may be located in a single location (e.g., within a home environment, an office environment or as a server farm), while in other examples the processors may be distributed across a number of locations.

The one or more processors may also operate to support performance of the relevant operations in a “cloud computing” environment or as a “software as a service” (SaaS). For example, at least some of the operations may be performed by a group of computers (as examples of machines including processors), with these operations being accessible via a network (e.g., the Internet) and via one or more appropriate interfaces (e.g., Application Program Interfaces (APIs)).

Example embodiments (e.g., apparatus, systems, or methods) may be implemented in digital electronic circuitry, in computer hardware, in firmware, in software, or in any combination thereof. Example embodiments may be implemented using a computer program product (e.g., a computer program, tangibly embodied in an information carrier or in a machine readable medium, for execution by, or to control the operation of, data processing apparatus such as a programmable processor, a computer, or multiple computers).

A computer program may be written in any form of programming language, including compiled or interpreted languages, and may be deployed in any form, including as a stand-alone program or as a software module, subroutine, or other unit suitable for use in a computing environment. A computer program may be deployed to be executed on one computer or on multiple computers at one site or distributed across multiple sites and interconnected by a communication network.

In an example, operations may be performed by one or more programmable processors executing a computer program to perform functions by operating on input data and generating output. Examples of method operations may also be performed by, and example apparatus can be implemented as, special purpose logic circuitry (e.g., a field programmable gate array (FPGA) or an application-specific integrated circuit (ASIC)).

The computing system or systems herein may include clients and servers. A client and server may generally be remote from each other and generally interact through a communication network. The relationship of client and server arises by virtue of computer programs running on the respective computers and having a client-server relationship to each other. In embodiments deploying a programmable computing system, it will be appreciated that both hardware and software architectures may be adapted, as appropriate. Specifically, it will be appreciated that whether to implement certain functionality in permanently configured hardware (e.g., an ASIC), in temporarily configured hardware (e.g., a combination of software and a programmable processor), or a combination of permanently and temporarily configured hardware may be a function of efficiency. Below are set out hardware (e.g., machine 400) and software architectures that may be implemented in or as example embodiments.

In an example, the machine 400 may operate as a standalone device or the machine 400 may be connected (e.g., networked) to other machines.

In a networked deployment, the machine 400 may operate in the capacity of either a server or a client machine in server-client network environments. In an example, machine 400 may act as a peer machine in peer-to-peer (or other distributed) network environments. The machine 400 may be a personal computer (PC), a tablet

PC, a set-top box (STB), a Personal Digital Assistant (PDA), a mobile telephone, a web appliance, a network router, switch or bridge, or any machine capable of executing instructions (sequential or otherwise) specifying actions to be taken (e.g., performed) by the machine 400. Further, while only a single machine 400 is illustrated, the term “machine” shall also be taken to include any collection of machines that individually or jointly execute a set (or multiple sets) of instructions to perform any one or more of the embodiments discussed herein.

Example machine (e.g., computer system) 400 may include a processor 402 (e.g., a central processing unit (CPU), a graphics processing unit (GPU) or both), a main memory 404 and a static memory 406, some or all of which may communicate with each other via a bus 408. The machine 400 may further include a display unit 410, an alphanumeric input device 412 (e.g., a keyboard), and a user interface (UI) navigation device 411 (e.g., a mouse). In an example, the display unit410, input device 412 and UI navigation device 414 may be a touch screen display. The machine 400 may additionally include a storage device (e.g., drive unit) 416, a signal generation device 418 (e.g., a speaker), a network interface device 420, and one or more sensors 421, such as a global positioning system (GPS) sensor, compass, accelerometer, or other sensor.

The storage device 416 may include a machine readable medium 422 on which is stored one or more sets of data structures or instructions 424 (e.g., software) embodying or utilized by any one or more of the methodologies or functions described herein. The instructions 424 may also reside, completely or at least partially, within the main memory 404, within static memory 406, or within the processor 402 during execution thereof by the machine 400. In an example, one or any combination of the processor 402, the main memory 404, the static memory 406, or the storage device 416 may constitute machine readable media.

While the machine readable medium 422 is illustrated as a single medium, the term “machine readable medium” may include a single medium or multiple media (e.g., a centralized or distributed database, and/or associated caches and servers) that may be configured to store the one or more instructions 424. The term “machine readable medium” may also be taken to include any tangible medium that may be capable of storing, encoding, or carrying instructions for execution by the machine and that cause the machine to perform any one or more of the embodiments of the present disclosure or that may be capable of storing, encoding or carrying data structures utilized by or associated with such instructions. The term “machine readable medium” may accordingly be understood to include, but not be limited to, solid-state memories, and optical and magnetic media. Specific examples of machine readable media may include non-volatile memory, including, by way of example, semiconductor memory devices (e.g., Electrically Programmable Read-Only Memory (EPROM), Electrically Erasable Programmable Read-Only Memory (EEPROM)) and flash memory devices; magnetic disks such as internal hard disks and removable disks; magneto-optical disks; and CD-ROM and DVD-ROM disks.

The instructions 424 may further be transmitted or received over a communications network 426 using a transmission medium via the network interface device 420 utilizing any one of a number of transfer protocols (e.g., frame relay, IP, TCP, UDP, HTTP, etc.). Example communication networks may include a local area network (LAN), a wide area network (WAN), a packet data network (e.g., the Internet), mobile telephone networks (e.g., cellular networks), Plain Old Telephone (POTS) networks, and wireless data networks (e.g., IEEE 802.11 standards family known as Wi-Fi®, IEEE 802.16 standards family known as WiMax®), peer-to-peer (P2P) networks, among others. The term “transmission medium” may include any intangible medium that may be capable of storing, encoding or carrying instructions for execution by the machine, and includes digital or analog communications signals or other intangible medium to facilitate communication of such software.

Although the present embodiments have been described in detail, those skilled in the art will understand that various changes, substitutions, variations, enhancements, nuances, gradations, lesser forms, alterations, revisions, improvements and knock-offs of the embodiments disclosed herein may be made without departing from the spirit and scope of the embodiments in their broadest form.

1. An artificial pancreas control system for regulating insulin infusion to a subject having Type 1 diabetes to minimize and/or prevent an occurrence of hypoglycemia in response to the subject engaging in exercise, the system comprising:
a prediction module configured to generate a prediction of glucose uptake for the subject; and
an insulin infusion control module configured to automatically generate a rate of basal insulin infusion, based on the prediction comprising a predetermined probability of exercise being engaged in by the subject, and to cause delivery of insulin to the subject according to the generated rate to maintain a glucose level thereof within an optimal range.

2. The artificial pancreas control system according to claim 1, wherein each of the prediction module and the insulin infusion module is included in at least one controller configured to communicate with a glucose monitoring device configured to transmit glucose levels of the subject and with an insulin delivery device configured to deliver insulin to the subject according to the generated rate.

3. The artificial pancreas control system according to claim 1, wherein the optimal range is between about 70 mg/dl and about 180 mg/dl.

4. The artificial pancreas control system according to claim 1, wherein the prediction is based on the Subcutaneous Oral Glucose Minimal Model.

5. The artificial pancreas control system according to claim 1, wherein the prediction module comprises at least one exercise profile for the subject that defines an exercise pattern.

6. The artificial pancreas control system according to claim 1, wherein the probability of engagement in exercise by the subject is determined as being positive according to a predetermined level of glucose uptake of the subject being determined as corresponding to the at least one exercise profile.

7. The artificial pancreas control system according to claim 1, wherein the at least one controller is configured to cause delivery of insulin to the subject according to the generated rate in advance of the subject engaging in the exercise pattern of the at least one exercise profile.

8. The artificial pancreas control system according to claim 1, wherein the insulin infusion control module is further configured to calculate an insulin bolus according to an amount of insulin uptake resulting from exercise by the subject according to the at least one exercise profile, and wherein the insulin infusion control module is further configured to adjust the generated rate in response to receipt of a meal announcement.

9. (canceled)

10. The artificial pancreas control system according to claim 7, wherein the controller is further configured to receive real-time signaling of the engagement in exercise by the subject, and to adjust the delivery of basal insulin according to a determined glucose level received by the controller from the glucose monitoring device at the time of the signaling, and wherein the insulin infusion control module is further configured to calculate an insulin bolus according to an amount of insulin uptake resulting from the subject engaging in the exercise corresponding to the real-time signaling.

11. (canceled)

12. A processor-implemented method for regulating insulin infusion to a subject having Type 1 diabetes and equipped with an insulin delivery device to minimize and/or prevent an occurrence of hypoglycemia in response to the subject engaging in exercise, the method comprising:
generating a dynamic model to predict glucose uptake for the subject, the model including at least one exercise profile for the subject that defines an exercise pattern therefor;
assigning a predetermined level of glucose uptake to the at least one exercise profile;
interpreting the dynamic model to determine whether the dynamic model includes a probability of the subject engaging in exercise according to the at least one exercise profile;
determining a glucose level of the subject based on readings generated by a glucose monitoring device in communication with the subject;
if the probability is positive, automatically adjusting a basal insulin infusion rate, via the insulin delivery device, to be within an optimal range.

13. The method according to claim 12, wherein the glucose monitoring device is a continuous glucose monitoring device.

14. The method according to claim 13, wherein the optimal range is between about 70 mg/dl and about 180 mg/dl.

15. The method according to claim 12, wherein the adjusting satisfies a cost function that weights a spread between amounts of two consecutive basal insulin injections, wherein the adjusting satisfies a cost function that weights a spread between a current glucose value and a future glucose value corresponding to the predetermined level of glucose uptake, and wherein the cost function applies a penalty for a glucose value corresponding to hypoglycemia.

16. (canceled)

17. (canceled)

18. The method according to claim 12, wherein the dynamic model is generated using a Kalman filter methodology.

19. The method according to claim 12, wherein the processor is programmable to communicate with the insulin delivery device in a closed-loop or an open-loop.

20. The method according to claim 12, further comprising adjusting the basal insulin infusion rate in response to the processor receiving a meal announcement.

21. The method of claim 12, further comprising calculating an insulin bolus according to an amount of insulin uptake resulting from the engagement in exercise by the subject.

22. The method of claim 12, wherein the processor is further configured to receive real-time signaling of the engagement in exercise by the subject, and to adjust the delivery of basal insulin according to a determined glucose level received by the processor from the glucose monitoring device at the time of the signaling.

23. The method of claim 12, wherein a plurality of processors automatically adjusts the basal insulin infusion rate, via the insulin delivery device, to be within the optimal range.

24. A non-transitory computer readable medium having stored thereon computer readable instructions according to claim 12.
